(19) United States
(12) Patent Application Publication
(10) Pub. No.: US 2024/0180947 A1
(43) Pub. Date: Jun. 6, 2024

(54) LYOPHILIZED FORMULATIONS OF CD73 COMPOUNDS

(71) Applicant: Arcus Biosciences, Inc., Hayward, CA (US)

(72) Inventors: Balaji AGORAM, Burlingame, CA (US); Eric F. CONNOR, San Jose, CA (US); Ramprakash GOVINDARAJAN, Navi Mumbai (IN); Manshiu LEUNG, Daly City, CA (US); Kai-Hsin LIAO, San Diego, CA (US); Meng-Chieh LIN, Belmont, CA (US); Andrew M. K. PENNELL, San Francisco, CA (US)

(21) Appl. No.: 18/490,434

(22) Filed: Oct. 19, 2023

Related U.S. Application Data
(60) Provisional application No. 63/380,357, filed on Oct. 20, 2022.

(51) Int. Cl. A61K 31/706; A61J 1/06; A61K 9/19; A61K 31/337; A61K 39/395; A61K 47/18; A61K 47/22; A61K 47/26; A61K 47/36; A61K 47/40; A61P 35/00; A61P 35/04

(52) U.S. Cl. CPC A61K 31/706; A61J 1/065; A61K 9/19; A61K 31/337; A61K 39/3955; A61K 47/183; A61K 47/22; A61K 47/26; A61K 47/36; A61K 47/40; A61P 35/00; A61P 35/04

(57) ABSTRACT

Lyophilized formulations comprising a compound of Formula (I)

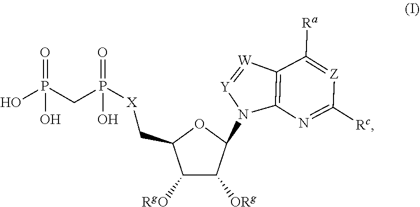

and one or more amino acids are provided, wherein W, X, Y, Z, R^{a}, R^{c}, and each R^{g} are as defined herein. Also provided are methods of preparation, methods of use, and dosage forms.

### CROSS-REFERENCE TO RELATED APPLICATIONS

This application claims the benefit to U.S. Provisional Application No. 63/380,357, filed Oct. 20, 2022, which is hereby incorporated by reference in its entirety.

### BACKGROUND

Ectonucleotides catalyze the conversion of ATP to adenosine, an endogenous modulator that impacts multiple systems, including the immune system, the cardiovascular system, the central nervous system, and the respiratory system. Adenosine also promotes fibrosis in a variety of tissues. In the first step of the production of adenosine, ectonucleoside triphosphate diphosphohydrolase 1 (ENTPD1), also known as CD39 (Cluster of Differentiation 39), hydrolyzes ATP to ADP, and then ADP to AMP. In the next step, AMP is converted to adenosine by 5′-nucleotidase, ecto (NTSE or 5NT), also known as CD73 (Cluster of Differentiation 73).

The enzymatic activities of CD39 and CD73 play strategic roles in calibrating the duration, magnitude, and chemical nature of purinergic signals delivered to various cells (e.g., immune cells). Alteration of these enzymatic activities can change the course or dictate the outcome of several pathophysiological events, including cancer, autoimmune diseases, infections, atherosclerosis, and ischemia-reperfusion injury, suggesting that these ecto-enzymes represent novel therapeutic targets for managing a variety of disorders.

The CD73 inhibitors in clinical development have been antibodies, as the development of small molecules has been hampered due to, for example, less than ideal metabolic and physical stability, as well as the availability of suitable formulations that can effectively deliver the agent.

In view of the role played by CD73 in cancer, as well as a diverse array of other diseases, disorders, and conditions, and the current lack of effective small molecule formulations of CD73 inhibitors available to medical practitioners, there is a need for such compositions and methods associated therewith.

### BRIEF SUMMARY

In one aspect, provided herein are lyophilized formulations comprising a compound of Formula (I)

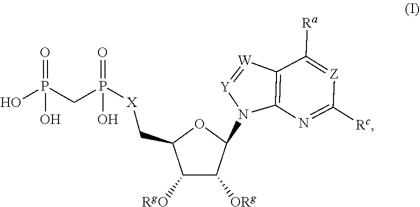

or a pharmaceutically acceptable salt thereof, and one or more amino acids, wherein W, X, Y, Z, R^{a}, R^{c}, and each R^{g} are as defined herein. Also provided are methods of preparation, methods of use, and dosage forms as described in detail below.

In one particular aspect, the present disclosure provides a lyophilized formulation of a compound having Formula (Ia):

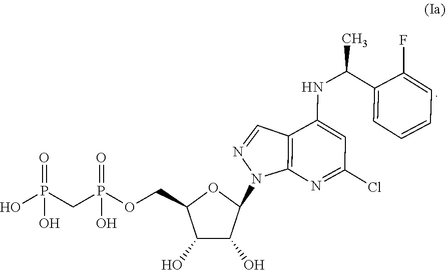 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]

The disclosure also provides an aqueous solution (e.g., a pre-lyophilized or a reconstituted lyophilized formulation) comprising a compound of Formula (I) or (Ia), one or more amino acids, a pH-adjusting agent, and, in some embodiments, a bulking agent. The present disclosure also relates to the use of the reconstituted lyophilized formulations for the treatment and/or prevention of a diverse array of diseases, disorders and conditions mediated, in whole or in part, by CD73. CD73 inhibitors have been linked to the treatment of a diverse array of disorders, including cancer, fibrosis, neurological and neurodegenerative disorders (e.g., depression and Parkinson's disease), cerebral and cardiac ischemic diseases, immune-related disorders, and disorders with an inflammatory component. See, e.g., Sorrentino et al (2013) OncoImmunol, 2:e22448, doi: 10.4161/onci.22448; and Regateiro et al. (2012) Clin. Exp. Immunol, 171:1-7. In particular embodiments, the formulations described herein can inhibit the immunosuppressive activity and/or the anti-inflammatory activity of CD73, and are useful as therapeutic or prophylactic therapy when such inhibition is desired. Unless otherwise indicated, when uses refer to a compound described herein, it is to be understood that such compound may be in any solid form (crystalline, amorphous, or mixtures thereof) or non-solid form.

In some embodiments, the present disclosure contemplates methods for treating or preventing cancer in a subject (e.g., a human) comprising administering to the subject a therapeutically effective amount of a compound of Formula (I) or (Ia) (e.g., as a reconstituted lyophilized formulation comprising a compound of Formula (I) or (Ia) described herein). The present disclosure includes methods of treating or preventing a cancer in a subject by administering to the subject a compound of Formula (I) or (Ia) (e.g., as a reconstituted lyophilized formulation described herein) in an amount effective to reverse, stop or slow the progression of CD73-mediated immunosuppression.

The present disclosure further contemplates the use of a compound of Formula (I) or (Ia) as described herein in combination with one or more additional agents. The one or more additional agents may have some CD73-modulating activity and/or they may function through distinct mechanisms of action. In some embodiments, such agents comprise radiation (e.g., localized radiation therapy or total body radiation therapy) and/or other treatment modalities of a non-pharmacological nature (e.g., surgical resection). In still other embodiments, when combination therapy is utilized, the reconstituted lyophilized formulation comprising a compound of Formula (I) or (Ia) as described herein and the one or more additional agent(s) may be in the form of a single composition or multiple compositions, and the treatment modalities can be administered concurrently, sequentially, or through some other regimen. By way of example, the present disclosure contemplates a treatment regimen wherein a radiation phase is followed by a chemotherapeutic phase or where treatment with a compound of Formula (I) or (Ia) for a period of time allows for subsequent surgical resection. The combination therapy can have an additive or synergistic effect. Other benefits of combination therapy are described hereafter.

### BRIEF DESCRIPTION OF THE DRAWINGS

FIG. 1 is a flow chart depicting an exemplary process for the bulk lyophilization of a formulation according to the disclosure. WFI=water for injection; qs=add sufficient quantity; raw material refers to excipients.

FIG. 2 shows a population PK simulation following doses at 50 mg QW (once weekly), 100 mg Q2W (every 2 weeks), and 300 mg Q3W (every 3 weeks). Solid line indicates population median; shaded areas indicate 5^{th} to 95^{th} quantiles of interindividual variability in predicted compound of Formula (Ia) concentrations; dashed line represents IC₉₀ of Formula (Ia) for inhibiting plasma CD73 enzymatic activities measured.

### DETAILED DESCRIPTION

Before the present disclosure is further described, it is to be understood that the disclosure is not limited to the particular embodiments set forth herein, and it is also to be understood that the terminology used herein is for the purpose of describing particular embodiments only, and is not intended to be limiting.

The term “about” as used herein has its original meaning of approximately and is to provide literal support for the exact number that it precedes, as well as a number that is near to or approximately the number that the term precedes. In general, the term “about” refers to the usual error range for the respective value readily known to the skilled person in this technical field. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

### General

The present disclosure is directed to lyophilized formulations of CD73 inhibitors (e.g., compounds of Formula (I), or other nucleoside or nucleotide compounds) in combination with one or more amino acids. Surprisingly, the noted combination prevents aggregation of the CD73 inhibitors. The lyophilized formulations according to this disclosure may have certain advantages such as reduced cracking, brittleness and/or shrinkage. In some embodiments, the lyophilized formulations of this disclosure are stable and can be stored at about 40° C./75% relative humidity (“RH”), or 25° C./60% RH, and 2-8° C. for up to 3 months until reconstitution. In some embodiments, the stored lyophiles reconstitute rapidly to yield clear solutions and the impurity profile of the reconstituted solutions is unchanged when compared to the initial lyophile. In some embodiments, the compound of Formula (I) is (((((2R,3S,4R,5R)-5-(6-chloro-4-(((S)-1-(2-fluorophenyl)ethyl)amino)-1H-pyrazolo[3,4-b]pyridin-1-yl)-3,4-dihydroxytetrahydrofuran-2-yl)methoxy)(hydroxy)phosphoryl)-methyl)phosphonic acid (also known as quemliclustat or AB680), shown below as Formula (Ia):

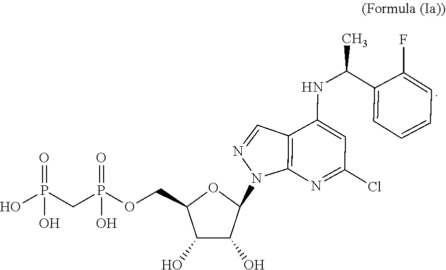 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]

### Definitions

Unless otherwise indicated, the following terms are intended to have the meaning set forth below. Other terms are defined elsewhere throughout the specification.

The term “alkyl,” by itself or as part of another substituent, means, unless otherwise stated, a saturated straight or branched chain hydrocarbon radical, having the number of carbon atoms designated (i.e. C₁₋₈ means one to eight carbons). Examples of alkyl groups include methyl, ethyl, n-propyl, isopropyl, n-butyl, t-butyl, isobutyl, sec-butyl, n-pentyl, n-hexyl, n-heptyl, n-octyl, and the like.

The term “cycloalkyl” refers to hydrocarbon rings having the indicated number of ring atoms (e.g., C₃₋₆ cycloalkyl) and being fully saturated or having no more than one double bond between ring vertices. “Cycloalkyl” is also meant to refer to bicyclic and polycyclic hydrocarbon rings such as, for example, bicyclo[2.2.1]heptane, bicyclo12.2.21octane, etc.

The term “heterocycloalkyl” refers to a cycloalkyl ring having the indicated number of ring vertices (or members) and having from one to five heteroatoms selected from N, O, and S, which replace one to five of the carbon vertices, and wherein the nitrogen and sulfur atoms are optionally oxidized, and the nitrogen atom(s) are optionally quaternized. The heterocycloalkyl may be a monocyclic, a bicyclic or a polycylic ring system. Non limiting examples of heterocycloalkyl groups include pyrrolidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrahydrothiophene, quinuclidine, and the like. A heterocycloalkyl group can be attached to the remainder of the molecule through a ring carbon atom or a ring heteroatom, when chemically permissible.

As used herein, a wavy line, “ [symbol] ,” that intersects a single, double, or triple bond in any chemical structure depicted herein, represents that the point of attachment of the single, double, or triple bond to the remainder of the molecule is through either one of the atoms that make up the single, double or triple bond. Additionally, a bond extending to the center of a ring (e.g., a phenyl ring) is meant to indicate attachment at any of the available ring vertices, i.e., such that attachment of the substituent to the ring results in a chemically stable arrangement.

As referred to herein, divalent components include either orientation (forward or reverse) of that component. For example, the group “—C(O)NH—” is meant to include a linkage in either orientation: —C(O)NH— or —NHC(O)—, and similarly, “—O—CH₂CH₂—” is meant to include both —O—CH₂CH₂— and —CH₂CH₂—O—.

The terms “alkoxy,” “alkylamino,” and “alkylthio” (or thioalkoxy) are used in their conventional sense, and refer to those alkyl groups attached to the remainder of the molecule via an oxygen atom, an amino group, or a sulfur atom, respectively. Additionally, for dialkylamino groups, the alkyl portions can be the same or different and can also be combined to form a 3-7 membered ring with the nitrogen atom to which each is attached. Accordingly, a group represented as dialkylamino or —NR^{a}R^{b} is meant to include piperidinyl, pyrrolidinyl, morpholinyl, azetidinyl, and the like.

The terms “arylalkyl” and “heteroarylalkyl” are used in their conventional sense, and refer to those groups wherein an aryl group or a heteroaryl group is attached remainder of the molecule via C₁-C₄ alkylene linker. An exemplary embodiment of “arylalkyl” is phenylmethyl (or benzyl). Similarly, an exemplary embodiment of “heteroarylalkyl” is, for example, 3-pyridylpropyl. When ‘optionally substituted’ is used to describe either of the terms “arylalkyl” or “heteroarylalkyl,” it is meant to refer to those groups wherein the aryl or heteroaryl portion is optionally substituted as in the definitions below, and the alkyl portion is optionally substituted as in the definitions below.

The terms “cycloalkylalkyl” and “heterocycloalkylalkyl” are used in their conventional sense, and refer to those groups wherein a cycloalkyl group or a heterocycloalkyl group is attached remainder of the molecule via C₁-C₄ alkylene linker. When ‘optionally substituted’ is used to describe either of the terms “cycloalkylalkyl” or “heterocycloalkylalkyl,” it is meant to refer to those groups wherein the cycloalkyl or heterocycloalkyl portion is optionally substituted as in the definitions below, and the alkyl portion is optionally substituted as in the definitions below.

The term “alkylene” refers to a straight or branched, saturated, hydrocarbon radical having one to four (e.g., C₁-C₄ alkylene) carbon atoms, and linking at least two other groups, i.e., a divalent hydrocarbon radical. When two moieties are linked to the alkylene they can be linked to the same carbon atom (i.e., geminal), e.g., —(CH(CH₃))—, or different carbon atoms of the alkylene group. For instance, a straight chain alkylene can be the bivalent radical of —(CH₂)ₙ-, where n is 1, 2, 3, or 4 (i.e., a C₁₋₄ alkylene). Representative alkylene groups include, but are not limited to, methylene, ethylene, propylene, isopropylene, butylene, isobutylene, secbutylene, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “C₁₋₄ haloalkyl” is meant to include trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon group which can be a single ring or multiple rings (up to three rings) which are fused together or linked covalently. In some embodiments, an aryl can have six to fourteen (i.e., C6-14 aryl), or six to ten (i.e., C6-10 aryl), or six (i.e., C6 aryl) carbon atoms. Non-limiting examples of aryl groups include phenyl, naphthyl, and biphenyl.

The term “heteroaryl” refers to aryl groups (or rings) that contain from one to five heteroatoms selected from N, O, and S, wherein the nitrogen and sulfur atoms are optionally oxidized, and the nitrogen atom(s) are optionally quaternized. In some embodiments, a heteroaryl” can have from 5 to 14 (i.e., 5- to 14-membered heteroaryl), or from 5 to 10 (i.e., 5- to 10-membered heteroaryl), or from 5 to 6 (i.e., 5- to 6-membered heteroaryl) members (i.e., ring vertices), and contain from one to five, one to four, one to three, one to two or one heteroatom selected from nitrogen (N), oxygen (O), and sulfur (S). A heteroaryl group can be attached to the remainder of the molecule through a ring carbon atom, or a ring heteroatom, when chemically permissible. Non-limiting examples of heteroaryl groups include pyridyl, pyridazinyl, pyrazinyl, pyrimindinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl, and the like. Substituents for a heteroaryl ring can be selected from the group of acceptable substituents described below.

The above terms (e.g., “alkyl,” “aryl,” and “heteroaryl”), in some embodiments, will be optionally substituted. Selected substituents for each type of radical are provided below.

Optional substituents for the alkyl radicals (including those groups often referred to as alkylene, alkenyl, alkynyl and cycloalkyl) can be a variety of groups selected from: halogen, —OR′, —NR′R″, —SR′, —SiR′R″R″′, —OC(O)R′, —C(O)R′, —CO₂R′,—CONR′R″, —OC(O)NR′R″, —NR″C(O)R′, —NR′—C(O)NR″R″′, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —NR′S(O)₂R″, —CN and —NO₂ in a number ranging from zero to (2 m′+1), where m′ is the total number of carbon atoms in such radical. R′, R″ and R″′ each independently refer to hydrogen, unsubstituted C₁₋₈ alkyl, unsubstituted aryl, aryl substituted with 1-3 halogens, unsubstituted C₁₋₈ alkyl, C₁₋₈ alkoxy or C₁₋₈ thioalkoxy groups, or unsubstituted aryl-C₁₋₄ alkyl groups. When R′ and R″ are attached to the same nitrogen atom, they can be combined with the nitrogen atom to form a 3-, 4-, 5-, 6-, or 7-membered ring. For example, —NR′R″ is meant to include 1-pyrrolidinyl and 4-morpholinyl.

Similarly, optional substituents for the aryl, heteroaryl, cycloalkyl, and heterocycloalkyl groups are varied and are generally selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R″′, —NH—C(NH₂)═NH, —NR′C(NH₂)═NH, —NH—C(NH₂)═NR′, —S(O)R′, —S(O)₂R′, —S(O)₂NR′R″, —NR′S(O)₂R″, —N₃, perfluoro(C₁-C₄)alkoxy, and perfluoro(C₁-C₄)alkyl, in a number ranging from zero to the total number of open valences on the aromatic ring system; and where R′, R″ and R″′ are independently selected from hydrogen, C₁₋₈ alkyl, C₁₋₈haloalkyl, C₃₋₆ cycloalkyl, C₂₋₈ alkenyl, and C₂₋₈ alkynyl. Other suitable substituents include each of the above aryl substituents attached to a ring atom by an alkylene tether of from 1-4 carbon atoms.

As used herein, the term “heteroatom” is meant to include oxygen (O), nitrogen (N), sulfur (S), and silicon (Si). In some embodiments, the heteroatom is O or N.

The term “pharmaceutically acceptable salts” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc, and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids, and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts. Additionally, certain compounds containing both acidic and basic functionalities may exist as a zwitterion (i.e., an inner salt).

The neutral forms of the compounds may be regenerated by contacting the salt with a base or acid and isolating the parent compound in the conventional manner The parent form of the compound differs from the various salt forms in certain physical properties, such as solubility in polar solvents, but otherwise the salts are equivalent to the parent form of the compound for the purposes of the present disclosure.

In addition to salt forms, the present disclosure provides compounds which are in a prodrug form. Prodrugs of the compounds described herein are those compounds that readily undergo chemical changes to provide the compounds of the present disclosure. Generally, a prodrug contains a moiety that is cleaved in vivo resulting in a compound of the present disclosure.

Certain compounds of the present disclosure can exist as unsolvated forms as well as solvated forms (e.g., ethanol or ethyl acetate solvates), including hydrated forms, all of which are intended to be encompassed within the scope of the present disclosure. Certain compounds of the present disclosure may exist in multiple crystalline or amorphous forms (see, e.g., WO 2020/123772 and WO 2021/257643). In general, all physical forms are contemplated by the present disclosure and are intended to be within the scope of the present disclosure.

Certain compounds of the present disclosure possess asymmetric carbon atoms (optical centers) or double bonds; the racemates, diastereomers, geometric isomers, regioisomers and individual isomers (e.g., separate enantiomers) are all intended to be encompassed within the scope of the present disclosure. When a stereochemical depiction is shown (e.g., using dashes, [symbol] , and/or wedges, [symbol] ), it is meant to refer the compound in which one of the isomers is present and substantially free of the other isomer. ‘Substantially free of’ another isomer indicates at least an 80/20 ratio of the two isomers, more preferably 90/10, or 95/5 or more. In some embodiments, one of the isomers will be present in an amount of at least 99%. A chemical bond to an asymmetric carbon that is depicted as a solid line ( [symbol] ) indicates that all possible stereoisomers (e.g., enantiomers, diastereomers, racemic mixtures, etc.) at that carbon atom are included.

“Hydrate” refers to a complex formed by, for example, combining compounds of Formula (I) (CD73 inhibiting compounds) and water. The term includes stoichiometric as well as non-stoichiometric hydrates.

“Solvate” refers to a complex formed by the combining of compounds of Formula (I) and a solvent. Exemplary solvents that form solvates include, but are not limited to methanol, methyl tert-butyl ether, ethanol, isopropanol, DMSO, ethyl acetate, acetic acid, and acetonitrile. In some embodiments, the solvate is an ethanol, ethyl acetate, or acetonitrile solvate.

“Desolvated” refers to a compound of Formula (I) that is a solvate as described herein, and from which solvent molecules have been partially or completely removed. Desolvation techniques to produce desolvated forms include, without limitation, exposure of a compound of Formula (I) (solvate) to a vacuum, subjecting the solvate to elevated temperature, exposing the solvate to a stream of gas, such as air or nitrogen, slurrying the solvate in a different solvent, or any combination thereof. Thus, a desolvated form of a compound of Formula (I) can be completely without solvent molecules, or partially solvated wherein solvent molecules are present in stoichiometric or non-stoichiometric amounts.

“Alcohol” refers to a solvent having a hydroxy group. Representative alcohols can have any suitable number of carbon atoms, such as C₁-C₆, and any suitable number of hydroxy groups, such as 1-3. Exemplary alcohols include, but are not limited to, methanol, ethanol, n-propanol, i-propanol, etc.

The terms “patient” or “subject” are used interchangeably to refer to a human or a non-human animal (e.g., a mammal).

The terms “treat,” “treating,” treatment,” and the like refer to a course of action (such as administering an inhibitor of CD73 or a pharmaceutical composition comprising same) initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, condition afflicting a subject. Thus, treatment includes inhibiting (e.g., arresting the development or further development of the disease, disorder or condition or clinical symptoms association therewith) an active disease, improving the quality of life, and/or prolonging survival of the subject. Treatment also refers to a course of action resulting in remission (whether partial or total) of the disease, disorder, or condition.

The term “in need of treatment” as used herein refers to a judgment made by a physician or other caregiver that a subject requires or will benefit from treatment. This judgment is made based on a variety of factors that are in the realm of the physician's or caregiver's expertise.

The terms “prevent,” “preventing,” “prevention,” and the like refer to a course of action (such as administering an CD73 inhibitor or a pharmaceutical composition comprising same) initiated in a manner (e.g., prior to the onset of a disease, disorder, condition or symptom thereof) so as to prevent, suppress, inhibit or reduce, either temporarily or permanently, a subject's risk of developing a disease, disorder, condition, or the like (as determined by, for example, the absence of clinical symptoms) or delaying the onset thereof, generally in the context of a subject predisposed to having a particular disease, disorder, or condition. In certain instances, the terms also refer to slowing the progression of the disease, disorder, or condition, or inhibiting progression thereof to a harmful or otherwise undesired state. Prevention also refers to a course of action initiated in a subject after the subject has been treated for a disease, disorder, condition, or symptom in order to prevent the relapse of that disease, disorder, condition, or symptom.

The term “in need of prevention” as used herein refers to a judgment made by a physician or other caregiver that a subject requires or will benefit from preventative care. This judgment is made based on a variety of factors that are in the realm of a physician's or caregiver's expertise.

The phrase “therapeutically effective amount” means a dose regimen (i.e., amount and interval) of the compound that provides the specific pharmacological effect for which the compound is administered to a subject in need of such treatment. For treatment, a therapeutically effective amount may be effective to reduce, ameliorate, or eliminate one or more signs or symptoms associated with a disease, delay disease progression, prolong survival, decrease the dose of other medication(s) required to treat the disease, or a combination thereof. With respect to cancer specifically, a therapeutically effective amount may, for example, result in the killing of cancer cells, reduce cancer cell counts, reduce tumor burden, eliminate tumors or metastasis, or reduce metastatic spread. A therapeutically effective amount may vary based on, for example, one or more of the following: the age and weight of the subject, the subject's overall health, the stage of the subject's disease, the route of administration, and prior or concomitant treatments. An “effective amount” with reference to a CD73 inhibitor of the present disclosure means an amount of the compound that is sufficient to engage the target (e.g., by inhibiting the target) at a level that is indicative of the potency of the compound. Target engagement can be determined by one or more biochemical or cellular assays resulting in an EC50, ED50, EC90, IC50, or similar value which can be used as one assessment of the potency of the compound.

The phrases “lyophilized formulation” and “lyophiles” are used interchangeably and refer to a composition that is formed by lyophilization; such formulations are further described herein.

The phrases “reconstituted lyophilized formulation” and “reconstituted formulation” and “reconstituted solution” and “reconstituted aqueous solution” and “reconstituted pharmaceutical composition” are used interchangeably, and refer to the composition that is formed when the lyophilized formulation according to this disclosure is reconstituted with a diluent.

“Substantially pure” indicates that a component makes up greater than about 50% of the total content of the composition, and typically greater than about 60% of the total content. More typically, “substantially pure” refers to compositions in which at least 75%, at least 85%, at least 90% or more of the total composition is the component of interest. In some cases, the component of interest will make up greater than about 90%, or greater than about 95% of the total content of the composition. In some embodiments, a compound of Formula (I) or (Ia) as described herein is substantially pure, i.e. at least 75%, at least 85%, at least 90% or more, or greater than about 90%, or greater than about 95%, of the provided material is the compound of Formula (I) or (Ia).

The term “response,” for example, of a cell, tissue, organ, or organism, encompasses a change in biochemical or physiological behavior, e.g., concentration, density, adhesion, or migration within a biological compartment, rate of gene expression, or state of differentiation, where the change is correlated with activation, stimulation, or treatment, or with internal mechanisms such as genetic programming In certain contexts, the terms “activation,” “stimulation,” and the like refer to cell activation as regulated by internal mechanisms, as well as by external or environmental factors; whereas the terms, “inhibition,” “down-regulation,” and the like refer to the opposite effects.

### I. Pre-Lyophilized Compositions

In one aspect, provided herein are aqueous compositions comprising a compound of Formula (I) or a pharmaceutically acceptable salt, hydrate, or solvate thereof, an amino acid, and a pH-adjusting agent sufficient to give the aqueous composition a pH between about 6 to about 8, wherein the compound of Formula (I) has the structure:

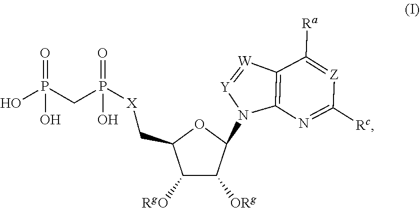

wherein W, X, Y, Z, R^{a}, R^{c}, and each R^{g} are as defined herein. In some embodiments, the composition further comprises a bulking agent as described elsewhere herein.
   Ia. Compounds of Formula (I)

Compounds useful in preparing the pre-lyophilized compositions and lyophilized formulations described herein are represented by Formula (I):

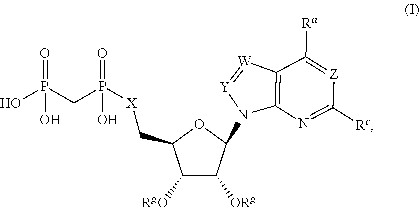

or a pharmaceutically acceptable salt, hydrate, or solvate thereof, wherein,
   W is selected from the group consisting of CR^{e} and N;
   X is selected from the group consisting of O, CH₂, and S;
   each of Y and Z is independently selected from the group consisting of CH and N:
   R^{g} is H or the two R^{g} groups are combined to form an acetonide;
   R^{a} is selected from the group consisting of NH₂, NHR¹, and NR¹R²;
   R^{c} is selected from the group consisting of H, halogen, haloalkyl, NH₂, NHR³, NR³R⁴, R³, OH, OR³, SR³, SO₂R³, —X¹—NH₂, —X¹—NHR³, —X¹—NR³R⁴, —X¹—OH, —X¹—OR³, —X¹—SR³, and —X¹—SO₂R³;
   R^{e} is selected from the group consisting of H, halogen, and optionally substituted C₁-C₆ alkyl;
   each X¹ is C₁-C₄ alkylene; and
   each R¹, R², R³, and R⁴ is independently selected from the group consisting of optionally substituted C₁-C₁₀ alkyl, optionally substituted C₃-C₇ cycloalkyl, optionally substituted C₃-C₇ cycloalkyl C₁-C₄ alkyl, optionally substituted 4-7 membered heterocycloalkyl, optionally substituted 4-7 membered heterocycloalkyl C₁-C₄ alkyl, optionally substituted aryl, optionally substituted aryl C₁-C₄ alkyl, optionally substituted heteroaryl, and optionally substituted heteroaryl C₁-C₄ alkyl, or when R¹ and R² or R³ and R⁴ are attached to the same nitrogen, they combine to form a 4- to 7-membered heterocyclic ring.

In some embodiments, the compound of Formula (I) is the compound wherein X is O.

In some embodiments, the compound of Formula (I) is the compound wherein each R^{g} is H.

In some embodiments, the compound of Formula (I) is the compound of any embodiments above, wherein R^{a} is NHR¹ and R¹ is selected from the group consisting of optionally substituted aryl C₁-C₄ alkyl and optionally substituted heteroaryl C₁-C₄ alkyl. In some embodiments, the optionally substituted aryl C₁-C₄ alkyl or optionally substituted heteroaryl C₁-C₄ alkyl is substituted with 1-3 halo.

In some embodiments, the compound of Formula (I) is the compound of any embodiments above, wherein R^{a} is NHR¹ and R¹ is optionally substituted aryl C₁-C₄ alkyl. In some embodiments, the optionally substituted aryl C₁-C₄ alkyl is substituted with 0-3 halo. In some embodiments, the optionally substituted aryl C₁-C₄ alkyl is substituted with 1-3 halo. In some embodiments, the optionally substituted aryl C₁-C₄alkyl is substituted with 1 fluoro.

In some embodiments, the compound of Formula (I) is the compound of any embodiments above, wherein R^{c} is selected from the group consisting of H, halogen, and haloalkyl.

In some embodiments, the compound of Formula (I) is the compound of any embodiments above, wherein R^{c} is halogen.

In some embodiments, the compound of Formula (I) is the compound wherein W is CR^{e}.

In some embodiments, the compound of Formula (I) is the compound of any embodiments above, wherein R^{e} is H.

In some embodiments, the compound of Formula (I) is the compound having a structure selected from the group consisting of:

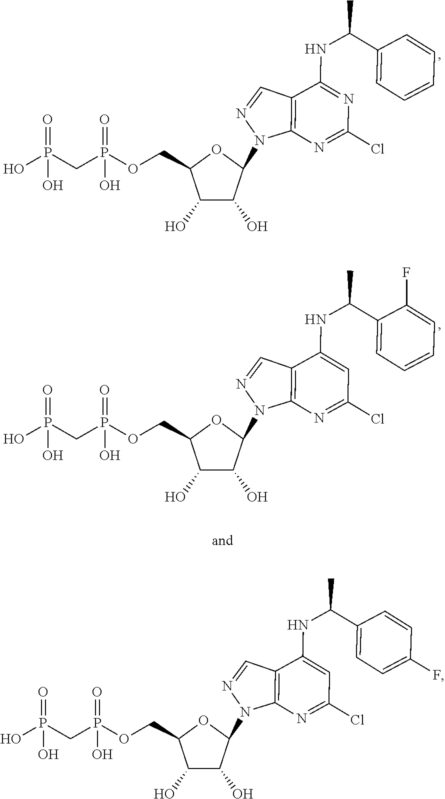 [SMILES: C[C@H](Nc1nc(Cl)nc2c1cnn2[C@@H]1O[C@H](COP(=O)(O)CP(=O)(O)O)[C@@H](O)[C@H]1O)c1ccccc1 ; C[C@H](Nc1cc(Cl)nc2c1cnn2[C@@H]1O[C@H](COP(=O)(O)CP(=O)(O)O)[C@@H](O)[C@H]1O)c1ccccc1F ; C[C@H](Nc1cc(Cl)nc2c1cnn2[C@@H]1O[C@H](COP(=O)(O)CP(=O)(O)O)[C@@H](O)[C@H]1O)c1ccc(F)cc1]

or a pharmaceutically acceptable salt thereof.

In some embodiments, the compound of Formula (I) is provided to a solution to form an aqueous composition as described herein. In one embodiment, the compound of Formula (I) is provided to the solution as a free acid. In another embodiment, the compound of Formula (I) is provided to the solution as a pharmaceutically acceptable salt.

In some embodiments, the compound of Formula (I) is a compound represented by Formula (Ia):

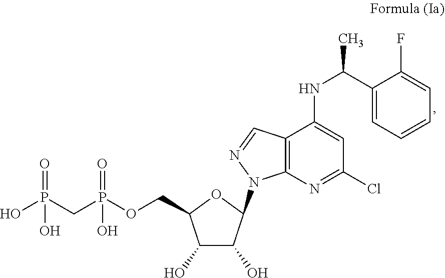 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]

and is provided as a free acid. In another embodiment, the compound of Formula (Ia) is provided to the solution as a pharmaceutically acceptable salt.

In some embodiments, the compound of Formula (I) has a structure according to Formula (Ia) or a pharmaceutically acceptable salt thereof.

In one embodiment, the compound of Formula (I), is a compound represented by Formula (Ia), and is provided in a crystalline form:

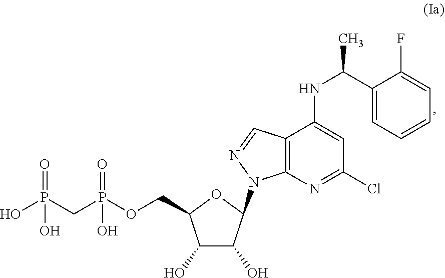 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]

wherein the crystalline form is any one of crystalline Forms I to VI, as described in W02021/257643, or crystalline Forms A-D as described in WO 2020/123772, each of which are hereby incorporated by reference.

In general, the compounds of Formula (I) are prepared using the methods described in, for example, WO 2017/120508; Org. Process Res. Dev. 2023, 27, 5, 945-953; and Org. Process Res. Dev. 2021, 25, 1, 157-162.

Ib. Amino Acid

The pre-lyophilized compositions and lyophilized formulations described herein also comprise one or more amino acids. The presence of the one or more amino acids in the compositions according to this disclosure improve the physical stability of the reconstituted lyophilized formulation. Without wishing to be bound by theory, the amphiphilic nature of the compounds described herein (i.e., the compounds of Formula (I) and Formula (Ia)) results in a tendency for the reconstituted solution to undergo a physical change attributable to aggregation of molecules. Accordingly, the presence of the one or more amino acids disrupts aggregation, offering the reconstituted compositions improved physical stability as compared to those lacking one or more amino acids. The amino acid is generally provided as an aqueous solution. In some embodiments, the amino acid is a basic amino acid. In yet another embodiment, the amino acid is selected from arginine, histidine, lysine, tryptophan, cysteine, or combinations thereof. In some embodiments, the one or more amino acids comprises arginine, lysine, histidine, tryptophan, cysteine, or a combination thereof. In some embodiments, the amino acid is arginine. In some embodiments, the amino acid is histidine. In some embodiments, the amino acid is lysine. In some embodiments, the amino acid is tryptophan. In some embodiments, amino acid is cysteine. In some embodiments, the amino acid is arginine, histidine, or a combination thereof. In some embodiments, the amino acid is a combination of arginine and histidine.

k. pH-Adjusting Agent

The pre-lyophilized compositions and lyophilized formulations described herein also make use of a pH-adjusting agent. The pH-adjusting agent may be a weak acid such as, by way of non-limiting example: acetic acid, citric acid, hydrochloric acid, propionic acid, tartaric acid, fumaric acid, lactic acid, phosphoric acid, and malic acid. In one embodiment, the pH-adjusting agent is phosphoric acid. Generally, the pH-adjusting agent is provided as an aqueous solution. While the concentration of the aqueous solution is not critical, the pH-adjusting agent is provided to the composition comprising the compound of Formula (I) or (Ia) and one or more amino acids in an amount sufficient to give the solution a pH in the range between about 6 to about 8. In another embodiment, the pH-adjusting agent is provided to the composition comprising the compound of Formula (I) or (Ia) and the one or more amino acids in an amount sufficient to give the solution a pH in the range between about 6.5 and about 7.5. In one embodiment, the pH-adjusting agent is phosphoric acid, and it is provided to the composition comprising Formula (I) or (Ia) and one or more amino acids in an amount sufficient to give the solution a pH in the range of 6.0-7.0. In one embodiment, the pH-adjusting agent is phosphoric acid, and it is provided to the composition comprising Formula (I) or (Ia), and one or more amino acids in an amount sufficient to give the solution a pH in the range of 7.0-7.5.

Id. Bulking Agent

In one or more embodiments, the pre-lyophilized compositions, lyophilized formulations, and aqueous solutions described herein comprise a bulking agent. Bulking agents form the bulk of the lyophilized formulation and provide an adequate structure to the lyophilized cake. Bulking agents can be used for low dose (or low concentration of) drugs that do not have the necessary bulk to support their own structure. The structure of the lyophilized cake is important, since proper cake formation leads to proper pore formation that provides a means for vapor to escape from the formulation during the drying cycle. Exemplary bulking agents include, but are not limited to mannitol, glycine, hydroxypropyl-Beta-cyclodextrin (HPBCD) (i.e., kleptose), dextran, sucrose, lactose, trehalose, sorbitol, glucose, raffinose, and the like, and are utilized in pre-lyophilized compositions to provide structure to the lyophilized cake, preventing cake collapse from occurring. In some embodiments, the bulking agent in the compositions and formulations according to this disclosure is mannitol, glycine, hydroxypropyl-Beta-cyclodextrin (HPBCD), or dextran. In one embodiment, the bulking agent in the compositions and formulations according to this disclosure is glycine. In one embodiment, the bulking agent in the compositions and formulations according to this disclosure is mannitol.

Ie. Vial Selection

Some embodiments provide for a vial containing a lyophilized formulation as described herein or an aqueous formulation as described herein.

In one or more embodiments, the vial used to lyophilize the pre-lyophilized formulation is a clear, glass vial (USP type 1). In one or more embodiments, the vial used to lyophilize the pre-lyophilized formulation is characterized by a hydrophobic coating on its inner surface. Accordingly, in some embodiments, a vial comprises an inner surface in contact with a hydrophobic coating. Exemplary vials include SCHOTT TopLyo® vials, or those described in U.S. Pat. No. 8,592,015, which is hereby incorporated by reference. The presence of a hydrophobic coating reduces the adherence of the compositions and/or formulations according to this disclosure to the side of the vial, and contributes to the formation of a lyophilized cake that is free of cracking, brittleness, and/or shrinkage. Reduced adherence of the compositions and/or formulations according to this disclosure to the side of the vial allows for ease of reconstitution which contributes to consistently accurate dosing of a compound according to this disclosure.

The hydrophobic coating can be applied to the inner surface of the vial using methods known to those skilled in the art, such as, for example, physical or chemical vapor phase deposition methods including, e.g., plasma-assisted chemical vapor deposition. In some embodiments, the hydrophobic coating on the inner surface of the vial comprises silicon (Si), oxygen (O), carbon (C), and hydrogen (H). In some embodiments, the hydrophobic coating is characterized by a low Si content. In some embodiments, the hydrophobic coating is characterized by an oxygen content to silicon content ratio of 1.2 or less. In some embodiments, the ratio of O to Si is less than or equal to 1.2. In some embodiments, the hydrophobic coating comprises a compound having the formula SiOₓC_{y}H_{z} wherein x is between 0.0 and 1.2; y is between 0.0 and 6.0; and z is between 0.0 and 6.0. In some embodiments, the hydrophobic coating comprises a compound having the formula SiOₓC_{y}Hₓ wherein x is between 0.6 and 0.9; y is between 1.2 and 3.3; and z is between 0.0 and 6.0. In some embodiments, the hydrophobic coating comprises a compound having the formula SiOₓC_{y}Hₓ wherein x is between 0.7 and 0.8; y is between 1.5 and 2.5; and z is between 0.0 and 6.0. In some embodiments, the hydrophobic coating is essentially free of fluorine. The composition of the hydrophobic coating can be determined using methods known to those skilled in the art, such as, for example, X-ray photoelectron spectroscopy (XPS).

The hydrophobicity of the coating can be determined by, for example, measuring the contact angle of a drop of water on the surface of the coating to determine a water contact angle. In some embodiments, the coated vial (i.e. the hydrophobic coating) is characterized by a water contact angle for water of greater than or equal to 90°. In some embodiments, the coated vial is characterized by a water contact angle of between about 90° and 120°, such as a contact angle of 90°, 95°, 100°, 105°, 110°, 115°, or 120°.

### II. Lyophilization Procedure

The process of lyophilization comprises three main stages: (1) freezing, (2) primary drying, and (3) secondary drying.

The freezing step ensures the pre-lyophilized composition is in a solid phase to form a frozen pre-lyophilized composition. Freezing is achieved by lowering and maintaining a temperature for a pre-determined amount of time. The material should be cooled to a temperature below the lowest melting point of all components present in the pre-lyophilized composition (including, e.g., a eutectic melting point that is lower than the melting point of any of the individual components) to freeze it and to ensure that sublimation rather than melting will occur with subsequent heating of the frozen material under vacuum or low pressure. Cooling the mixture to a temperature below the lowest temperature where the solid and liquid phase of the material can coexist (e.g., the eutectic point) ensures that sublimation rather than melting will occur in the following steps.

In some embodiments, the pre-lyophilized composition is cooled to a temperature lower than 0° C. In some embodiments, the pre-lyophilized composition is cooled to a temperature of −50° C. or lower. In some embodiments, the pre-lyophilized composition is cooled to a temperature of −40° C. to −50° C. In some embodiments, the pre-lyophilized composition is cooled to a temperature of about −45° C. In some embodiments, the pre-lyophilized composition is cooled for 10 hours or less. In some embodiments, the pre-lyophilized composition is be cooled for 9 hours or less, 8 hours or less, 7 hours or less, 6 hours or less, 5 hours or less, 4 hours or less, 3 hours or less, 2 hours or less, 1 hour or less, or 30 minutes or less.

There are a number of suitable methods for freezing, each is embraced within this disclosure. Suitable freezing methods include, but are not limited to refrigeration, placement in an ice bath (e.g., mixtures of ice or dry ice and a salt, or organic solvent, or combinations thereof, e.g., placement in a bath of dry ice with methanol or ethanol), and placement in a liquid nitrogen bath.

After freezing, the primary drying step includes lowering the pressure to induce sublimation of the frozen water in the frozen pre-lyophilized composition. Usually, most of the water is removed in this primary drying phase.

Typically, the pressure is lowered using a vacuum pump. The desired pressure may vary based on the temperature of the primary drying step, since the vapor pressure over ice is a function of temperature. In some embodiments, the pressure in the primary drying step is lowered below the vapor pressure of ice at the target temperature (i.e., the temperature of the primary drying step). For example, the vapor pressure of ice at −26° C. is about 429 mTorr. In the described embodiments, the pressure in the primary drying step is below 429 mTorr. In some embodiments, the pressure in the primary drying step is 20% to 70% of the vapor pressure of ice at the target temperature (i.e., the temperature of the primary drying step). Using −26° C. as the exemplary temperature again, 20% to 30% of the vapor pressure would be about 85.8 to 300 mTorr. In some embodiments, the pressure in the primary drying step is 20% to 30% of the vapor pressure of ice at the target temperature (i.e., the temperature of the primary drying step). With reference to the −26° C. as the exemplary temperature again, 20% to 30% of the vapor pressure would be about 85.8 to 128 mTorr. Vapor pressure over ice is generally known and can be accessed by a person of skill in the art. Exemplary vapor pressures at given temperatures include 0° C., about 4,584 mTorr; −10° C., about 1,949 mTorr; −20° C., about 774 mTorr; −30° C., about 285 mTorr; −40° C., about 96 mTorr; or −50° C., about 30 mTorr.

The primary drying step can be performed at a temperature at, below, or above ambient temperature. For example, in some embodiments, the primary drying step is performed at a temperature of 40° C. or less, 30° C. or less, 20° C. or less, 10° C. or less, or 0° C. or less.

The primary drying phase lasts for any suitable amount of time. In some embodiments, the primary drying phase is at least 30 minutes, 1 hour, 2 hours 3 hours, 4 hours, 5 hours, 6 hours, 7 hours, 8 hours, 9 hours, 10 hours or longer. In some embodiments, the primary drying phase is run overnight. In some embodiments, the primary drying temperature is −25° C., with warming to 25° C. for about 3 hours.

Following the primary drying phase, further drying (the secondary drying phase) is done by raising the temperature higher than the temperature used in the primary drying phase. Most of the free water is removed during the primary drying phase, leaving mostly water that is bound to components of the formulation. Thus, the secondary drying phase removes additional water content as well as water that has condensed or moved from an initial location during the primary drying phase. Since most of the free water is already removed, the temperature of the secondary drying phase can be increased without melting the composition.

In some embodiments, the secondary drying phase is performed at a temperature ranging from −10° C. to 50° C., from 0° C. to 40° C., from 10° C. to 30° C., or from 20° C. to 25° C.

Low pressure is typically used in this phase as well. Typically, the lowered pressure from the primary drying phase is maintained, but a variety of lower pressures that facilitate sublimation are suitable during this step.

The rate of water removal during the secondary drying phase is a function of temperature and lower pressures do not typically increase the drying rate. In some embodiments, the secondary drying phase is at least 30 minutes, 1 hour, 2 hours, 3 hours, 4 hours, 5 hours, 6 hours, 7 hours, 8 hours, 9 hours, 10 hours or longer. In some embodiments, the secondary drying phase is run overnight.

In some embodiments, the secondary drying phase can include drying in the presence of an inert gas (e.g., nitrogen) or a combination of inert gasses. For example, the lyophilization vessel and/or the vial storage container can be purged with an inert gas and capped to avoid exposure of the formation to the air.

The lyophilized compositions described here, after one or more drying steps, can have a moisture content of, for example, less than 20%. In some examples, the moisture content of the lyophilized compositions described herein are less than 15%, less than 10%, less than 5%, less than 4%, less than 3%, less than 2%, less than 1%, less than 0.5%, or less than 0.1%.

### III. Lyophilized Formulations Comprising Formula (I)

In one aspect, the disclosure provides a lyophilized formulation of a compound of Formula (I):

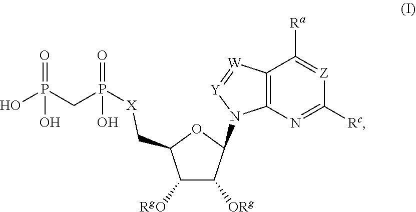

wherein W, X, Y, Z, R^{a}, R^{c}, and each R^{g} are as defined herein, or a pharmaceutically acceptable salt thereof, as described above in Section Ia, and one or more amino acids. In some embodiments, the one or more amino acids is an L-amino acid (e.g., L-arginine, etc.). In some embodiments, the lyophilized formulation further comprises a pH-adjusting agent, a bulking agent, or both.

In some embodiments, the lyophilized formulation has a molar ratio of the compound of Formula (I) (e.g., a compound of Formula (Ia)) to the one or more amino acids that is from about 1:2 to about 1:7. In some embodiments, the lyophilized formulation has a molar ratio of the compound of Formula (I) (e.g., a compound of Formula (Ia)) to the one or more amino acids that is from about 1:2 to about 1:5. In another embodiment, the molar ratio of the compound of Formula (I) (e.g., a compound of Formula (Ia)) to the amino acid is from about 1:4 to about 1:5. In another embodiment, the molar ratio of the compound of Formula (I) (e.g., a compound of Formula (Ia)) to the amino acid is about 1:4.5.

The lyophilized formulations may also be characterized by percent composition. In one embodiment, the lyophilized formulation comprises a compound of Formula (I) (e.g., a compound of Formula (Ia)), one or more amino acids, and a pH adjusting agent, and the compound of Formula (I) makes up about 35% to 45% by weight of the lyophilized formulation, the amino acid makes up about 45% to about 60% by weight of the lyophilized formulation, and the pH-adjusting agent make up about 5% to about 10% by weight of the lyophilized composition. In another embodiment, the lyophilized formulation comprises a compound of Formula (I) (e.g., a compound of Formula (Ia)), one or more amino acids, a pH adjusting agent, and a bulking agent, and the compound of Formula (I) or (Ia) makes up about 15% to about 25% by weight of the lyophilized formulation, the amino acid makes up about 20% to about 35% by weight of the lyophilized formulation, the pH-adjusting agent makes up about 1% to about 10% by weight of the lyophilized formulation, and the bulking agent makes up about 40% to about 50% by weight of the lyophilized formulation.

The lyophilized formulations according to this disclosure may have certain advantages such as reduced cracking, brittleness and/or shrinkage. Such characteristics may be determined according to methods known in the art.

In some embodiments, provided herein is a lyophilized formulation comprising a compound of Formula (Ia) or a pharmaceutically acceptable salt thereof, and one or more amino acids selected from arginine, lysine, histidine, tryptophan, cysteine, and combinations thereof in an amount greater than the stoichiometric amount of the compound of Formula (Ia).

In some embodiments, provided herein is a lyophilized formulation comprising

about 15% to about 20% by weight of a compound of Formula (Ia)

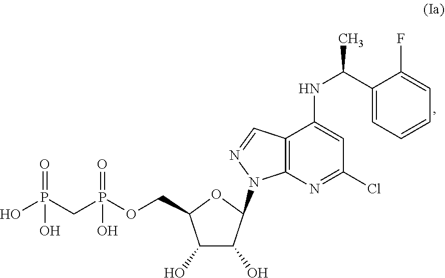 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]

or a pharmaceutically acceptable salt, hydrate, or solvate thereof,

about 20% to about 35% by weight of one or more amino acids;

about 2% to about 5% by weight of phosphoric acid; and

about 40% to about 60% by weight of a bulking agent;

wherein percentage by weight (i.e. % by weight) is based on the total weight of the lyophilized formulation.

In some embodiments, the bulking agent is kleptose, dextran, mannitol, or glycine. In some embodiments, the bulking agent is mannitol. In some embodiments, the bulking agent is glycine.

In some embodiments, provided herein is a lyophilized formulation comprising

about 17.4% by weight of the compound of Formula (Ia), or a pharmaceutically acceptable salt, hydrate, or solvate thereof,

about 23.4% by weight of arginine;

about 3.5% by weight of phosphoric acid; and

about 55.6% by weight of mannitol;
wherein percentage by weight is based on the total weight of the lyophilized formulation.

In some embodiments, provided herein is a lyophilized formulation comprising

about 22.6% by weight of the compound of Formula (Ia), or a pharmaceutically acceptable salt, hydrate, or solvate thereof,

about 30.6% by weight of arginine;

about 4.6% by weight of phosphoric acid; and

about 42.1% by weight of glycine;
wherein percentage by weight is based on the total weight of the lyophilized formulation.

In some embodiments, provided herein is a lyophilized formulation comprising

about 35% to about 45% by weight of a compound of Formula (Ia)

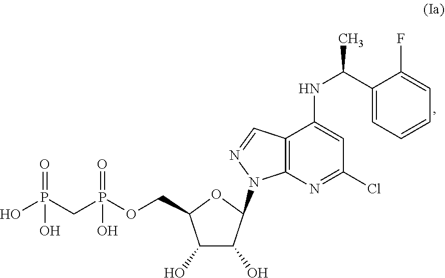 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]

or a pharmaceutically acceptable salt, hydrate, or solvate thereof,

about 45% to about 55% by weight of one or more amino acids;

about 5% to about 10% by weight of phosphoric acid; and
wherein percentage by weight (% by weight) is based on the total weight of the lyophilized formulation.

In some embodiments, provided herein is a lyophilized formulation comprising

about 39.2% by weight of the compound of Formula (Ia), or a pharmaceutically acceptable salt, hydrate, or solvate thereof,

about 52.9% by weight of arginine; and

about 7.9% by weight of phosphoric acid;
wherein percentage by weight is based on the total weight of the lyophilized formulation.

In some embodiments, the pH of the lyophilized formulation is adjusted by a pH-adjusting agent. It is contemplated that a skilled artisan would understand that the amount of any pH-adjusting agent (e.g., a base, such as sodium hydroxide, or an acid, such as phosphoric acid) present in a lyophilized formulation is determined, among other things, by the targeted pH-range prior to lyophilization. In some embodiments, the targeted pH range is between about 6 and about 8 (e.g., pH 7.0-7.5 or pH 7.2-7.4). In some embodiments, the lyophilized formulations has a pH of from about 7.2 to about 7.4. In some embodiments, the pH-adjusting agent comprise sodium hydroxide. In some embodiments, the amount of sodium hydroxide corresponds to the amount of sodium hydroxide added to a pre-lyophilization solution such that the pH of the pre-lyophilization solution is between about 6 and about 8; in some embodiments, between about 7.0 and about 7.5; and in some embodiments, between about 7.2 and about 7.4.

In some embodiments, provided herein is a lyophilized formulation in a vial comprising about 107.5 mg of a compound of Formula (Ia)

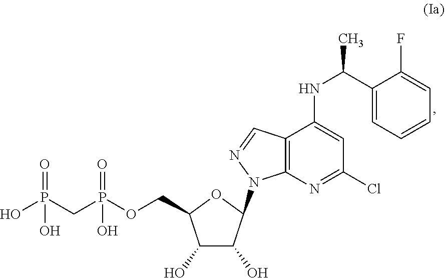 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]

or a pharmaceutically acceptable salt, hydrate, or solvate thereof,

about 145.34 mg of arginine;

about 21.8 mg of phosphoric acid; and

about 344 mg of mannitol.

In some embodiments, provided herein is a lyophilized formulation in a vial comprising

about 107.5 mg of a compound of Formula (Ia)

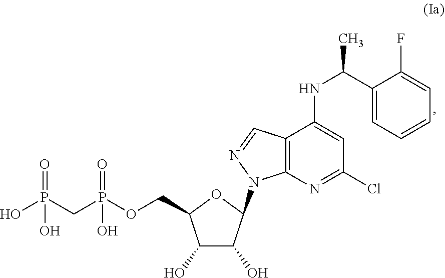 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]

or a pharmaceutically acceptable salt, hydrate, or solvate thereof,

about 145.34 mg of arginine;

about 21.8 mg of phosphoric acid; and

about 200 mg of glycine.

In some embodiments, provided herein is a lyophilized formulation in a vial comprising

about 27.5 mg of a compound of Formula (Ia)

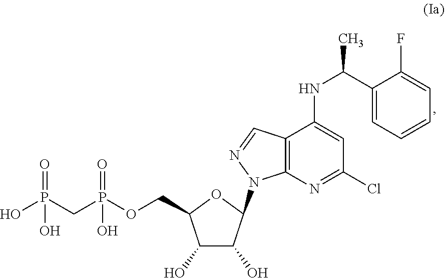 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]

or a pharmaceutically acceptable salt, hydrate, or solvate thereof,

about 37.1 mg of arginine; and

about 5.6 mg of phosphoric acid.

In some embodiments, provided herein is a lyophilized formulation in a vial comprising

about 107.5 mg of a compound of Formula (Ia)

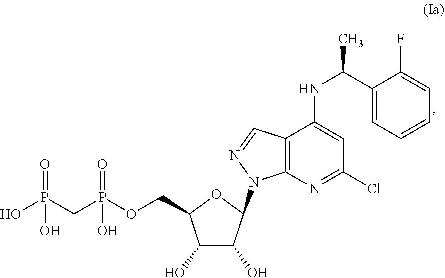 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]

or a pharmaceutically acceptable salt, hydrate, or solvate thereof,

about 145.1 mg of arginine; and

about 21.8 mg of phosphoric acid.

In some embodiments, the lyophilized formulation in a vial further comprises sodium hydroxide. In some embodiments, the amount of sodium hydroxide corresponds to the amount of sodium hydroxide added to a pre-lyophilization solution such that the pH of the pre-lyophilization solution is between about 6 and about 8; in some embodiments, between about 7.0 and about 7.5; and in some embodiments, between about 7.2 and about 7.4.

In some embodiments, a lyophilized formulation described herein is prepared by

(i) preparing a bulk solution by dissolving the compound of Formula (Ia), an amino acid (e.g., arginine, e.g., L-arginine), phosphoric acid, and a bulking agent in sterile water;

(ii) optionally adjusting the pH of the bulk solution by adding a pH-adjusting agent;

(iii) filtering the bulk solution into sterilized vials in a clean room;

(iv) loading the vials into a freeze-dryer;

(v) subjecting the vials to freeze-thaw cycles; and

(vi) subjecting the vials to primary and secondary drying under reduced pressure; to obtain the lyophilized formulation.

In some embodiments, a lyophilized formulation described herein is prepared by

(i) preparing a bulk solution by dissolving the compound of Formula (Ia), arginine, phosphoric acid, and a bulking agent in sterile water;

(ii) optionally adjusting the pH of the bulk solution by adding a pH-adjusting agent;

(iii) filtering the bulk solution into sterilized vials in a clean room;

(iv) loading the vials into a freeze-dryer;

(v) cooling the vials to a temperature of about −45° C. at the rate of about 0.5° C. per minute and holding the vials at the temperature of about −45° C. for about 3 hours;

(vi) warming the vials to a temperature of about −10° C. over about 50 minutes and holding the vials at the temperature of about −10° C. for about 4 hours;

(vii) cooling the vials to a temperature of about −45° C. over about 70 minutes and holding the vials at the temperature of about −45° C. for about 2 hours;

(viii) warming the vials to a temperature of about −15° C. and reducing the pressure and starting the primary drying;

(ix) maintaining the vials under reduced pressure for about 36 hours;

(x) warming the vials to a temperature of about 30° C. and conducting secondary drying for about 4 hours; and

(xi) backfilling the freeze-dryer with dry nitrogen to a pressure of about 700 Torr and stoppering the vials, thereby providing the lyophilized formulation.

In some embodiments, a lyophilized formulation described herein is prepared by

(i) preparing a bulk solution by dissolving the compound of Formula (Ia), an amino acid (e.g., arginine, e.g., L-arginine), and phosphoric acid in sterile water;

(ii) optionally adjusting the pH of the bulk solution by adding a pH-adjusting agent;

(iii) filtering the bulk solution into sterilized vials in a clean room;

(iv) loading the vials into a freeze-dryer;

(v) subjecting the vials to freeze-thaw cycles; and

(vi) subjecting the vials to primary and secondary drying under reduced pressure, thereby obtaining the lyophilized formulation.

In some embodiments, a lyophilized formulation described herein is prepared by

(i) preparing a bulk solution by dissolving the compound of Formula (Ia), arginine, and phosphoric acid in sterile water;

(ii) optionally adjusting the pH of the bulk solution by adding a pH-adjusting agent;

(iii) filtering the bulk solution into sterilized vials in a clean room;

(iv) loading the vials into a freeze-dryer;

(v) cooling the vials to a temperature of about −45° C. at the rate of about 0.5° C. per minute and holding the vials at the temperature of about −45° C. for about 3 hours;

(vi) warming the vials to a temperature of about −10° C. over about 50 minutes and holding the vials at the temperature of about −10° C. for about 4 hours;

(vii) cooling the vials to a temperature of about −45° C. over about 70 minutes and holding the vials at the temperature of about −45° C. for about 2 hours;

(viii) warming the vials to a temperature of about −15° C. and reducing the pressure and starting the primary drying;

(ix) maintaining the vials under reduced pressure for about 36 hours;

(x) warming the vials to a temperature of about 30° C. and conducting secondary drying for about 4 hours; and

(xi) backfilling the freeze-dryer with dry nitrogen to a pressure of about 700 Torr and stoppering the vials, thereby providing the lyophilized formulation.

In some embodiments, the pH-adjusting agent is a base. In some embodiments, the pH-adjusting agent is an acid. In some embodiments, the pH-adjusting agent is added to an aqueous solution that is subsequently subjected to lyophilization such that the pre-lyophilization aqueous solution has a pH between about 6 and about 8; in some embodiments, between about 7.0 and about 7.5; and in some embodiments between about 7.2 and 7.4.

The temperatures, times, pressures, and/or ranges of weight percentages noted above are only provided by way of example, other variations can be conceived by one of skill in the art and all such variations are contemplated within the scope of embodiments presented herein.

### IV. Reconstituting Lyophilized Formulations Comprising Formula (I)

In one aspect, this disclosure provides a reconstituted lyophilized formulation (i.e., a reconstituted pharmaceutical composition or reconstituted formulation) comprising a compound of Formula (I) or (Ia), one or more amino acids, and an acceptable diluent. In some embodiments, the acceptable diluent is a pharmaceutically acceptable diluent. In some embodiments, the pharmaceutically acceptable diluent is suitable for intravenous administration. In some embodiments, the reconstituted lyophilized formulation further comprises a pH-adjusting agent, a bulking agent, or both. The reconstituted lyophilized formulations (i.e., reconstituted pharmaceutical compositions) according to this disclosure are useful in methods of treating a disease, disorder, or condition mediated, at least in part, by CD73 as described elsewhere herein.

The lyophilized formulations described herein are reconstituted with an acceptable diluent prior to administration. “Reconstitution,” as used herein, refers to a process of wetting the lyophilized cake in a manner wherein the cake is completely dissolved, and the resulting liquid has reached acceptable clarity for administration to a patient. Diluents useful for reconstituting the lyophilized formulations comprising Formula (I) or (Ia) include sterile water for injection (SFWI), SWFI containing a stabilizer, a solubilizer, a tonicity modifier, such as NaCl, MgCl₂, or CaCl₂ etc., and mixtures thereof, 0.9% saline solution (i.e., normal saline), half normal saline, lactated Ringer's solution, dextrose in water, dextrose in saline, or dextrose in lactated Ringer's solution. In one embodiment, the diluent is SWFI. In another embodiment, the diluent is normal saline. In some embodiments, for example, where the vial containing the lyophilized formulation has been stored under refrigeration, the vial should be allowed to equilibrate to room temperature prior to reconstitution.

The volume of diluent used to reconstitute the formulations of the disclosure is dependent on the intended mode of administration, and the desired final concentration (mg/mL) of the reconstituted formulation. In some embodiments, the composition in the vial is reconstituted with 1.1 to 3.3 mL of diluent. One of skill in the art will appreciate that as vial size (and sample size) increases, the amount of diluent can also increase. Accordingly, in some embodiments, the composition in the vial is reconstituted with 2.2 to 20.2 mL of diluent, such as 2.2 mL, 2.3 mL, 2.4 mL, 2.5 mL, 2.6 mL, 2.7 mL, 2.8 mL, 2.9 mL, 3.0 mL, 3.1 mL, 3.2 mL, 3.3 mL, 3.4 mL, 3.5 mL, 3.6 mL, 3.7 mL, 3.8 mL, 3.9 mL, 4.0 mL, 4.1 mL, 4.2 mL, 4.3 mL, 4.4 mL, 4.5 mL, 4.6 mL, 4.7 mL, 4.8 mL, 4.9 mL, 5.0 mL, 5.1 mL, 5.2 mL, 5.3 mL, 5.4 mL, 5.5 mL, 5.6 mL, 5.7 mL, 5.8 mL, 5.9 mL, 6.0 mL, 6.1 mL, 6.2 mL, 6.3 mL, 6.4 mL, 6.5 mL, 6.6 mL, 6.7 mL, 6.8 mL, 6.9 mL, 7.0 mL, 7.1 mL, 7.2 mL, 7.3 mL, 7.4 mL, 7.5 mL, 7.6 mL, 7.7 mL, 7.8 mL, 7.9 mL, 8.0 mL, 8.1 mL, 8.2 mL, 8.3 mL, 8.4 mL, 8.5 mL, 8.6 mL, 8.7 mL, 8.8 mL, 8.9 mL, 9.0 mL, 9.1 mL, 9.2 mL, 9.3 mL, 9.4 mL, 9.5 mL, 9.6 mL, 9.7 mL, 9.8 mL, 9.9 mL, 10.0 mL, 10.1 mL, 10.2 mL 10.3 mL, 10.4 mL, 10.5 mL, 10.6 mL, 10.7 mL, 10.8 mL, 10.9 mL, 11.0 mL, 11.1 mL, 11.2 mL, 11.3 mL, 11.4 mL, 11.5 mL, 11.6 mL, 11.7 mL, 11.8 mL, 11.9 mL, 12.0 mL, 12.1 mL, 12.2 mL, 12.3 mL, 12.4 mL, 12.5 mL, 12.6 mL, 12.7 mL, 12.8 mL, 12.9 mL, 13.0 mL, 13.1 mL, 13.2 mL, 13.3 mL, 13.4 mL, 13.5 mL, 13.6 mL, 13.7 mL, 13.8 mL, 13.9 mL, 14.0 mL, 14.1 mL, 14.2 mL, 14.3 mL, 14.4 mL, 14.5 mL, 14.6 mL, 14.7 mL, 14.8 mL, 14.9 mL, 15.0 mL, 15.1 mL, 15.2 mL, 15.3 mL, 15.4 mL, 15.5 mL, 15.6 mL, 15.7 mL, 15.8 mL, 15.9 mL, 16.0 mL, 16.1 mL, 16.2 mL, 16.3 mL, 16.4 mL, 16.5 mL, 16.6 mL, 16.7 mL, 16.8 mL, 16.9 mL, 17.0 mL , 17.1 mL, 17.2 mL, 17.3 mL, 17.4 mL, 17.5 mL, 17.6 mL, 17.7 mL, 17.8 mL, 17.9 mL, 18.0 mL, 18.1 mL, 18.2 mL, 18.3 mL, 18.4 mL, 18.5 mL, 18.6 mL, 18.7 mL, 18.8 mL, 18.9 mL, 19.0 mL, 19.1 mL, 19.2 mL, 19.3, 19.4, 19.5, 19.6, 19.7, 19.8 mL, 19.9 mL, or 20.0 mL of diluent. In one embodiment, the lyophilized formulations described herein can be reconstituted in the diluent in about 15 minutes or less. In other embodiments, the lyophilized formulations comprising Formula (I) or (Ia) can be reconstituted in 10 minutes or less, or 5 minutes or less, or 4 minutes or less, or 3 minutes or less, or 2 minutes or less, or 1 minute or less.

In some embodiments, the diluent is water and provides an aqueous formulation described herein.

Provided herein is an aqueous formulation comprising a compound of Formula (I)

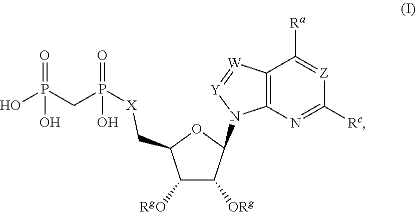

or a pharmaceutically acceptable salt, hydrate, or solvate thereof,
wherein,
W is selected from the group consisting of CR^{e} and N;
X is selected from the group consisting of O, CH₂, and S;
each of Y and Z is independently selected from the group consisting of CH and N:
R^{g} is H or the two R^{g} groups are combined to form an acetonide;
R^{a} is selected from the group consisting of NH₂, NHR¹, and NR¹R²;
   R^{c} is selected from the group consisting of H, halogen, haloalkyl, NH₂, NHR³, NR³R⁴, R³, OH, OR³, SR³, SO₂R³, —X¹—NH₂, —X¹—NHR³, —X¹—NR³R⁴, —X¹—OH, —X¹—OR³, —X¹—SR³, and —X¹—SO₂R³;
R^{e} is selected from the group consisting of H, halogen, and optionally substituted C₁-C₆ alkyl;
each X¹ is C₁-C₄ alkylene; and
each R¹, R², R³, and R⁴ is independently selected from the group consisting of optionally substituted C₁-C₁₀ alkyl, optionally substituted C₃-C₇ cycloalkyl, optionally substituted C₃ -C₇ cycloalkyl C₁-C₄ alkyl, optionally substituted 4-7 membered heterocycloalkyl, optionally substituted 4-7 membered heterocycloalkyl C₁-C₄ alkyl, optionally substituted aryl, optionally substituted aryl C₁-C₄ alkyl, optionally substituted heteroaryl, and optionally substituted heteroaryl C₁-C₄ alkyl, or when R¹ and R² or R³ and R⁴ are attached to the same nitrogen, they combine to form a 4- to 7-membered heterocyclic ring;
one or more amino acids and optionally a pH-adjusting agent in an amount sufficient to adjust the pH of the solution to between about 6 and about 8; in some embodiments, between about 7.0 and about 7.5; and in some embodiments between about 7.2 and about 7.4.

Provided herein is an aqueous formulation comprising

about 15% to about 20% by weight of a compound of Formula (Ia)

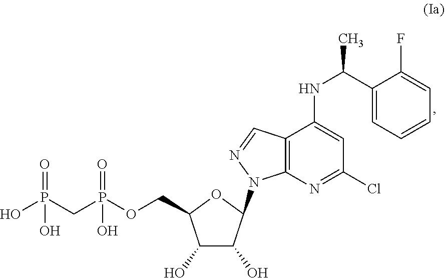 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]

or a pharmaceutically acceptable salt, hydrate, or solvate thereof,

about 20% to about 35% by weight of one or more amino acids;

about 2% to about 5% by weight of phosphoric acid; and

about 40% to about 60% by weight of a bulking agent;
wherein percentage by weight (% by weight) is based on the total weight of a lyophilized formulation that is dissolved in water to form the aqueous formulation.

In some embodiments, the bulking agent is kleptose, dextran, mannitol, or glycine. In some embodiments, the bulking agent is mannitol. In some embodiments, the bulking agent is glycine.

In some embodiments, provided is an aqueous formulation, comprising

about 17.4% by weight of the compound of Formula (Ia), or a pharmaceutically acceptable salt, hydrate, or solvate thereof,

about 23.4% by weight of arginine;

about 3.5% by weight of phosphoric acid; and

about 55.6% by weight of mannitol.
wherein percentage by weight is based on the total weight of a lyophilized formulation that is dissolved in water to form the aqueous formulation. In some embodiments, the lyophilized formulation is any lyophilized formulation described herein.

In some embodiments, provided is an aqueous formulation, comprising

about 22.6% by weight of the compound of Formula (Ia), or a pharmaceutically acceptable salt, hydrate, or solvate thereof,

about 30.6% by weight of arginine;

about 4.6% by weight of phosphoric acid; and

about 42.1% by weight of glycine;
wherein percentage by weight is based on the total weight of a lyophilized formulation that is dissolved in water to form the aqueous formulation.

In some embodiments, provided herein is an aqueous formulation comprising

about 35% to about 45% by weight of a compound of Formula (Ia)

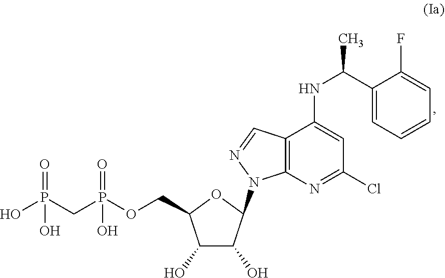 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]

or a pharmaceutically acceptable salt, hydrate, or solvate thereof,

about 45% to about 55% by weight of one or more amino acids;

about 5% to about 10% by weight of phosphoric acid; and
wherein percentage by weight is based on the total weight of a lyophilized formulation that is dissolved in water to form the aqueous formulation.

In some embodiments, provided herein is an aqueous formulation comprising

about 39.2% by weight of the compound of Formula (Ia), or a pharmaceutically acceptable salt, hydrate, or solvate thereof,

about 52.9% by weight of arginine; and

about 7.9% by weight of phosphoric acid;
wherein % by weight is based on the total weight of a lyophilized formulation that is dissolved in water to form the aqueous formulation.

In some embodiments, provided is an aqueous formulation comprising

about 107.5 mg of a compound of Formula (Ia)

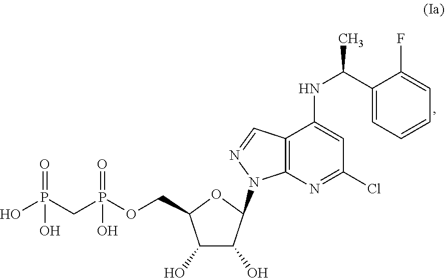 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]

or a pharmaceutically acceptable salt, hydrate, or solvate thereof,

about 145.34 mg of arginine;

about 21.8 mg of phosphoric acid; and

about 344 mg of mannitol.

In some embodiments, provided is an aqueous formulation comprising

about 107.5 mg of a compound of Formula (Ia)

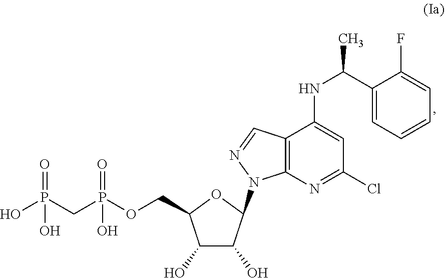 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]

or a pharmaceutically acceptable salt, hydrate, or solvate thereof,

about 145.34 mg of arginine;

about 21.8 mg of phosphoric acid; and

about 200 mg of glycine.

In some embodiments, provided herein is an aqueous formulation comprising

about 27.5 mg of a compound of Formula (Ia)

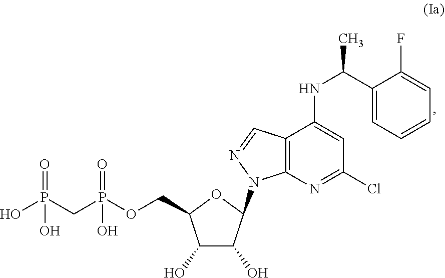 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]

or a pharmaceutically acceptable salt, hydrate, or solvate thereof,

about 37.1 mg of arginine; and

about 5.6 mg of phosphoric acid.

In some embodiments, provided herein is an aqueous formulation comprising

about 107.5 mg of a compound of Formula (Ia)

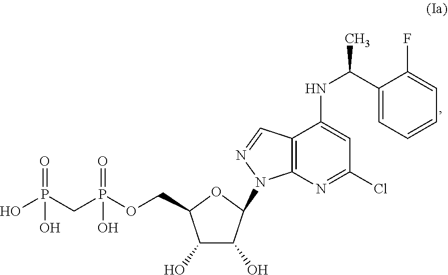 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]

or a pharmaceutically acceptable salt, hydrate, or solvate thereof,

about 145.1 mg of arginine; and

about 21.8 mg of phosphoric acid.

In some embodiments, the aqueous formulation further comprises a pH-adjusting agent, optionally wherein the pH-adjusting agent is sodium hydroxide. In some embodiments, the amount of sodium hydroxide corresponds to the amount of sodium hydroxide added to the aqueous solution such that the pH of the aqueous solution is between about 6 and about 8; in some embodiments, between about 7.0 and about 7.5; and in some embodiments, between about 7.2 and about 7.4.

### V. Therapeutic and Prophylactic Uses

In one aspect, this disclosure is directed to a method of treating a disease, disorder or condition mediated at least in part by CD73, said method comprising:

a) reconstituting a lyophilized formulation according to this disclosure with a diluent to form a reconstituted solution; and

b) administering a therapeutically effective amount of the reconstituted solution to a subject in need thereof.

The lyophilized formulation can be reconstituted with an acceptable diluent. In some embodiments, the diluent is selected from the group consisting of normal saline, half normal saline, Ringer's solution, lactated Ringer's solution, sterile water for injection, dextrose in water, dextrose in saline, and dextrose in lactated Ringer's solution. In one embodiment, the diluent is sterile water for injection. In another embodiment, the diluent is normal saline.

In some embodiments, the lyophilized formulation is reconstituted with from 2 to 20 mL of diluent. In some embodiments, the lyophilized formulation is reconstituted with from 2.0 to 15.0 mL of diluent. In some embodiments, the lyophilized formulation is reconstituted with from 2.0 to 10.0 mL of diluent. In some embodiments, the lyophilized formulation is reconstituted with 2.2 to 6.6 mL of diluent. In some embodiments, the lyophilized formulation is reconstituted with 3.0 to 6.0 mL of diluent. In some embodiments, the lyophilized formulation is reconstituted with 3.0 to 4.0 mL of diluent. In some embodiments, the lyophilized formulation is reconstituted with 3.0 to 3.5 mL of diluent. In some embodiments, the lyophilized formulation is reconstituted with 6.5 to 10.5 mL of diluent. In some embodiments, the lyophilized formulation is reconstituted with 7.0 to 10.0 mL of diluent. In some embodiments, the lyophilized formulation is reconstituted with 7.5 to 9.0 mL of diluent. In some embodiments, the lyophilized formulation is reconstituted with 8.0 to 9.0 mL of diluent. In some embodiments, the lyophilized formulation is reconstituted with 8.3 to 8.8 mL of diluent.

In one or more embodiments, the pH of the reconstituted solution is in the range of about 6 to about 8. In some embodiments, the pH of the reconstituted solution is in the range of about 6.5 to about 7.5. In some embodiments, the pH of the reconstituted solution is in the range of about 6.0 to about 7.0. In some embodiments, the pH of the reconstituted solution is in the range of about 7.0 to about 7.5.

In some embodiments, the reconstituted solution is administered to the subject parenterally. In some embodiments, said parenteral administration is intravenous, intraperitoneal, subcutaneous, intradermal, or intramuscular. In some embodiments, said parenteral administration is intravenous.

In some embodiments, the method further comprises diluting the reconstituted solution with an acceptable vehicle to form a diluted reconstituted solution prior to administration to the subject. In some embodiments, the vehicle is selected from the group consisting of normal saline, half normal saline, Ringer's solution, lactated Ringer's solution, sterile water for injection, dextrose in water, dextrose in saline, and dextrose in lactated Ringer's solution. In some embodiments, the vehicle is normal saline.

In some embodiments, the diluted reconstituted solution is administered to the subject within 24 hours of dilution, such as within 24 hours, such as 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, 1.5 hours, 1.25 hours, 1 hour, 50 minutes, 45 minutes, 40 minutes, 35 minutes, 30 minutes, 25 minutes, 20 minutes, 15 minutes, 14 minutes, 13 minutes, 12 minutes, 11 minutes, 10 minutes, 9 minutes, 8 minutes, 7 minutes, 6 minutes, 5 minutes, 4 minutes, 3 minutes, 2 minutes, or 1 minute of dilution. In some embodiments, the diluted reconstituted solution is administered to the subject within 24 hours of dilution. In some embodiments, the diluted reconstituted solution is administered to the subject within 4 hours of dilution. In some embodiments, the diluted reconstituted solution is administered to the subject within 1 hour of dilution. In some embodiments, the diluted reconstituted solution is administered to the subject within 30 minutes of dilution.

In some embodiments, the method further comprises filtering the diluted reconstituted solution prior to administering to a subject. In one embodiment, the diluted reconstituted solution is filtered using an in-line filter.

The present disclosure contemplates the use of a compound of Formula (I) or (Ia) (e.g., a reconstituted formulation, e.g., reconstituted solution or diluted reconstituted solution as described herein) in the treatment or prevention of a broad range of diseases, disorders and/or conditions, and/or the symptoms thereof. The reconstituted formulation is appropriate for administration by injection. Common injection types include intravenous, subcutaneous and intramuscular; infusions are typically given intravenously. Other locations of application of parenteral administration include: epidural, intraspinal, intrathecal, intracerebral, intraarticular, intracardiac, intradermal, intraperitoneal, intravitreal, intraarterial, intraorbital, and transtracheal. While particular uses are described in detail hereafter, it is to be understood that the present disclosure is not so limited. Furthermore, although general categories of particular diseases, disorders and conditions are set forth hereafter, some of the diseases, disorders and conditions may be a member of more than one category, and others may not be a member of any of the disclosed categories. Oncology-related Disorders. In accordance with the present disclosure, the compound of Formula (I) or (Ia) (e.g., the reconstituted lyophilized formulation as described herein) can be used to treat or prevent a proliferative condition or disorder, including a cancer, for example, cancer of the uterus, cervix, breast, prostate, testes, gastrointestinal tract (e.g., esophagus, oropharynx, stomach, small or large intestines, colon, or rectum), kidney, renal cell, bladder, bone, bone marrow, skin, head or neck, liver, gall bladder, heart, lung, pancreas, salivary gland, adrenal gland, thyroid, brain (e.g., gliomas), ganglia, central nervous system (CNS) and peripheral nervous system (PNS), and cancers of the hematopoietic system and the immune system (e.g., spleen or thymus). The present disclosure also provides methods of treating or preventing other cancer-related diseases, disorders or conditions, including, for example, immunogenic tumors, non-immunogenic tumors, dormant tumors, virus-induced cancers (e.g., epithelial cell cancers, endothelial cell cancers, squamous cell carcinomas and papillomavirus), adenocarcinomas, lymphomas, carcinomas, melanomas, leukemias, myelomas, sarcomas, teratocarcinomas, chemically-induced cancers, metastasis, and angiogenesis. The disclosure contemplates reducing tolerance to a tumor cell or cancer cell antigen, e.g., by modulating activity of a regulatory T-cell and/or a CD8+ T-cell (see, e.g., Ramirez-Montagut, et al. (2003) Oncogene 22: 3180-87; and Sawaya, et al. (2003) New Engl. J. Med. 349: 1501-09). In particular embodiments, the tumor or cancer is pancreatic cancer, colorectal cancer, ovarian cancer, uterine cancer, breast cancer, gastroesophageal cancer, urothelial cancer, gastric cancer, melanoma, lung cancer, kidney cancer, liver cancer, glioblastoma, head and neck cancer, or leukemia. In some embodiments, the compound of Formula (I) or (Ia) (e.g., the reconstituted lyophilized formulation as described herein) is used to treat the cancer in a first-line setting (e.g., treatment naïve in the setting). In some embodiments, the compound of Formula (I) or (Ia) (e.g., the reconstituted lyophilized formulation as described herein) is used to treat the cancer in a second-line or greater setting. The use of the term(s) cancer-related diseases, disorders and conditions is meant to refer broadly to conditions that are associated, directly or indirectly, with cancer, and includes, e.g., angiogenesis and precancerous conditions such as dysplasia.

In some embodiments, the cancer is castrate resistant prostate cancer (CRPC), pancreatic ductal adenocarcinoma (PDAC), non-small cell lung cancer, renal cell carcinoma, or colorectal cancer. In some embodiments, the cancer is castrate resistant prostate cancer (CRPC), pancreatic ductal adenocarcinoma (PDAC), non-small cell lung cancer (NSCLC), clear cell renal cell carcinoma (ccRCC), or colorectal cancer (CRC). In some embodiments, the cancer is metastatic. In some embodiments, the cancer is non-small cell lung cancer. In some embodiments, the cancer is non-small cell lung cancer and the subject is treatment-naive. In some embodiments, the cancer is non-small cell lung cancer and the subject has disease progression in a prior line of therapy. In some embodiments, the cancer is pancreatic cancer, optionally metastatic pancreatic cancer. In some embodiments, the pancreatic cancer is pancreatic adenocarcinoma. In some embodiments, the cancer is metastatic pancreatic cancer and the subject is treatment-naive. In some embodiments, the cancer is metastatic pancreatic cancer. In some embodiments, the prior line of therapy comprises chemotherapy. In some embodiments, the subject is treatment-naive. In some embodiments, the subject has disease progression in a prior line of therapy. In some embodiments, the prior line of therapy comprises a checkpoint inhibitor, optionally wherein the checkpoint inhibitor is a PD-L1 antagonist or a PD-1 antagonist. In some embodiments, the prior line of therapy comprises chemotherapy and a checkpoint inhibitor, optionally wherein the checkpoint inhibitor is a PD-L1 antagonist or a PD-1 antagonist.

In certain embodiments, a cancer may be metastatic or at risk of becoming metastatic, or may occur in a diffuse tissue, including cancers of the blood or bone marrow (e.g., leukemia). In some further embodiments, the reconstituted lyophilized formulations described herein can be used to overcome T-cell tolerance.

In some embodiments, the present disclosure provides methods for treating and/or preventing a proliferative condition, cancer, tumor, or precancerous condition with the compound of Formula (I) or (Ia) (e.g., the reconstituted lyophilized formulations described herein) and at least one additional therapeutic or diagnostic agent, examples of which are set forth elsewhere herein.

In one embodiment, the cancer is a gastrointestinal malignancy such as pancreatic cancer. In one embodiment, the cancer is metastatic pancreatic adenocarcinoma. In one embodiment, a patient is treated for pancreatic cancer using the reconstituted formulations described herein and an anti-PD-1 antibody. In another embodiment, a patient is treated for pancreatic cancer, optionally pancreatic adenocarcinoma, using the reconstituted formulations described herein in combination with chemotherapy. In another embodiment, a patient is treated for metastatic pancreatic cancer using the reconstituted formulations described herein in combination with chemotherapy and optionally an anti-PD-1 antibody or an anti-PD-L1 antibody. In some embodiments, suitable chemotherapy regimens are described in the National Cancer Comprehensive Network (NCCN) Clinical Practice Guidelines for Pancreatic Adenocarcinoma (Version 2.2023). In some embodiments, the chemotherapy comprises (a) paclitaxel or nab-paclitaxel and (b) gemcitabine. In some embodiments, the chemotherapy comprises FOLFIRINOX (folinic acid, which is sometimes called calcium folinate or leucovorin, fluorouracil (5FU), irinotecan, and oxaliplatin). In some embodiments, the chemotherapy comprises gemcitabine, capecitabine, or 5-fluorouracil (5-FU). In another embodiment, a patient is treated for first line metastatic pancreatic cancer using a compound of Formula (I) or (Ia) and an anti-PD-1 antibody and standard of care agents for pancreatic cancer such as those described herein. For example, in some embodiments, a patient may be treated for first line metastatic pancreatic cancer using a compound of Formula (I) or (Ia) and an anti-PD-1 antibody and a gemcitabine and nab-paclitaxel chemotherapy regimen. In some embodiments, a patient may be treated for first line metastatic pancreatic cancer using a compound of Formula (I) or (Ia) and an anti-PD-1 antibody and a FOLFIRINOX chemotherapy regimen. Patients may be treatment experienced or treatment naïve.

In some embodiments, the methods described herein may be indicated as first line, second line or third line treatments. In some embodiments, the methods described herein are indicated as a first line treatment. In some embodiments, the methods described herein are indicated as a second line treatment. In some embodiments, the methods described herein are indicated as a third line treatment.

Immune-related Disorders and Disorders with an Inflammatory Component. As used herein, terms such as “immune disease,” “immune condition,” “immune disorder,” “inflammatory disease,” “inflammatory condition,” “inflammatory disorder” and the like are meant to broadly encompass any immune-related condition (e.g., an autoimmune disease) or a disorder with an inflammatory component that can be treated and/or prevented using a compound of Formula (I) or (Ia) (e.g., the reconstituted lyophilized formulation as described herein) such that some therapeutic benefit is obtained. Such conditions frequently are inextricably intertwined with other diseases, disorders and conditions. By way of example, an “immune condition” may refer to proliferative conditions, such as cancer, tumors, and angiogenesis; including infections (acute and chronic), tumors, and cancers that resist eradication by the immune system.

The compound of Formula (I) or (Ia) (e.g., the reconstituted lyophilized formulations described herein) can be used to increase or enhance an immune response; to improve immunization, including increasing vaccine efficacy; and to increase inflammation. Immune deficiencies associated with immune deficiency diseases, immunosuppressive medical treatment, acute and/or chronic infection, and aging can be treated using the compounds disclosed herein. The reconstituted lyophilized formulations described herein can also be used to stimulate the immune system of patients suffering from iatrogenically-induced immune suppression, including those who have undergone bone marrow transplants, chemotherapy, or radiotherapy.

In particular embodiments of the present disclosure, the compound of Formula (I) or (Ia) (e.g., the reconstituted lyophilized formulations described herein) is used to increase or enhance an immune response to an antigen by providing adjuvant activity. In a particular embodiment, at least one antigen or vaccine is administered to a subject in combination with the reconstituted lyophilized formulations described herein to prolong an immune response to the antigen or vaccine. Therapeutic compositions are also provided which include at least one antigenic agent or vaccine component, including, but not limited to, viruses, bacteria, and fungi, or portions thereof, proteins, peptides, tumor-specific antigens, and nucleic acid vaccines, in combination with the reconstituted lyophilized formulations described herein.

Selection of Patients. In some instances, the methods according to this disclosure may be indicated in certain patients, for example, based on CD73 as a biomarker, high microsatellite instability, or high tumor mutational burden. In some instances, the subject is identified as having an oncogene driven or oncogene addicted cancer that has a mutation in at least one gene associated with CD73. Methods of testing determining CD73 levels and the presence of CD73 associated oncogenes are disclosed in WO 2020/185859 and WO 2020/205527.

### Combination Therapy

The present disclosure contemplates the use of a compound of Formula (I) or (Ia) (e.g., as a reconstituted lyophilized formulation as described herein) alone or in combination with one or more additional therapy. Each additional therapy can be an active therapeutic agent or another treatment modality. In embodiments comprising one or more additional therapeutic agents, each agent may target a different, but complementary, mechanism of action. The additional therapeutic agents can be small chemical molecules; macromolecules such as proteins, antibodies, peptibodies, peptides, DNA, RNA or fragments of such macromolecules; or cellular or gene therapies. Non-limiting examples of additional treatment modalities include surgical resection of a tumor, bone marrow transplant, radiation therapy, and photodynamic therapy. The use of a compound of Formula (I) or (Ia) (e.g., as a reconstituted lyophilized formulation as described herein) in combination with one or more additional therapy can have a synergistic therapeutic or prophylactic effect on the underlying disease, disorder, or condition. In addition or alternatively, the combination therapy may allow for a dose reduction of one or more of the agents, thereby ameliorating, reducing or eliminating adverse effects associated with one or more of the agents. The compound of Formula (I) or (Ia) of the present disclosure may also be useful in overcoming adenosine-dependent immunosuppression, leading to enhanced therapeutic efficacy of other agents. Furthermore, such combination therapy may have a synergistic therapeutic or prophylactic effect on the underlying disease, disorder, or condition.

In embodiments comprising one or more additional treatment modality, the compound of Formula (I) or (Ia) (e.g., as a reconstituted lyophilized formulation as described herein) can be administered before, after, or during treatment with the additional treatment modality. In embodiments comprising one or more additional therapeutic agent, the therapeutic agents used in such combination therapy can be formulated as a single composition or as separate compositions. If administered separately, each therapeutic agent in the combination can be given at or around the same time, or at different times. Furthermore, the therapeutic agents are administered “in combination” even if they have different forms of administration (e.g., oral capsule and intravenous), they are given at different dosing intervals, one therapeutic agent is given at a constant dosing regimen while another is titrated up, titrated down or discontinued, or each therapeutic agent in the combination is independently titrated up, titrated down, increased or decreased in dosage, or discontinued and/or resumed during a patient's course of therapy. If the combination is formulated as separate compositions, in some embodiments, the separate compositions are provided together in a kit.

In some embodiments, the one or more additional therapeutic agent is a signal transduction inhibitor. As used herein, the term “signal transduction inhibitor” refers to an agent that selectively inhibits one or more steps in a signaling pathway. Signal transduction inhibitors (STIs) contemplated by the present disclosure include: (i) BCR-ABL kinase inhibitors (e.g., GLEEVEC®); (ii) epidermal growth factor receptor tyrosine kinase inhibitors (EGFR TKIs), including small molecule inhibitors (e.g., gefitinib, erlotinib, afatinib, and osimertinib), and anti-EGFR antibodies; (iii) inhibitors of the human epidermal growth factor (HER) family of transmembrane tyrosine kinases, e.g., HER-2/neu receptor inhibitors (e.g., HERCEPTIN®), and HER-3 receptor inhibitors; (iv) vascular endothelial growth factor receptor (VEGFR) inhibitors including small molecule inhibitors (e.g., axitinib, regorafenib, sunitinib and sorafenib), VEGF kinase inhibitors (e.g., lenvatinib, cabozantinib, pazopanib, tivozanib, XL092, etc.), anti-VEGF antibodies (e.g., bevacizumab), and anti-VEGFR antibodies (e.g., ramucirumab); (v) inhibitors of AKT family kinases or the AKT pathway (e.g., rapamycin); (vi) inhibitors of serine/threonine-protein kinase B-Raf (BRAF), such as, for example, vemurafenib, dabrafenib and encorafenib; (vii) inhibitors of rearranged during transfection (RET), including, for example, selpercatinib and pralsetinib; (viii) tyrosine-protein kinase Met (MET) inhibitors (e.g., tepotinib, tivantinib, cabozantinib and crizotinib); (ix) anaplastic lymphoma kinase (ALK) inhibitors (e.g., ensartinib, ceritinib, lorlatinib, crizotinib, and brigatinib); (x) inhibitors of the RAS signaling pathway (e.g., inhibitors of KRAS, HRAS, RAF, MEK, ERK) as described elsewhere herein; (xi) FLT-3 inhibitors (e.g., gilteritinib);(xii) inhibitors of Trop-2, such as, for example, the antibody drug conjugate sacituzumab govitecan-hziy; (xiii) inhibitors of the JAK/STAT pathway, e.g., JAK inhibitors including tofacitinib and ruxolitinib, or STAT inhibitors such as napabucasin; (xiv) inhibitors of NF-κB; (xv) cell cycle kinase inhibitors (e.g., flavopiridol); (xvi) phosphatidyl inositol kinase (PI3K) inhibitors; (xix) protein kinase B (AKT) inhibitors (e.g., capivasertib, miransertib); (xx) platelet-derived growth factor receptor (PDGFR) inhibitors (e g , imatinib, sunitinib, regorafenib, avapritinib, lenvatinib, nintedanib, famitinib, ponatinib, axitinib, repretinib, etc.); and (xxi) insulin-like growth factor receptor (IGFR) inhibitors (e.g., erlotinib, afatinib, gefitinib, osimertinib, dacomitinib). Agents involved in immunomodulation can also be used in combination with the compounds and formulations described herein for the suppression of tumor growth in cancer patients. In one or more embodiments, the additional therapeutic agent comprises an inhibitor of EGFR, VEGFR, HER-2, HER-3, BRAF, RET, MET, ALK, RAS (e.g., KRAS, MEK, ERK), FLT-3, JAK, STAT, NF-κB, PI3K, AKT, or any combinations thereof. Agents involved in immunomodulation can also be used in combination with a compound of Formula (I) or (Ia) (e.g., as a reconstituted lyophilized formulation comprising a compound of Formula (I) or (Ia) described herein) for the suppression of tumor growth in cancer patients.

In some embodiments, the one or more additional therapeutic agent is a chemotherapeutic agent. Examples of chemotherapeutic agents include, but are not limited to, alkylating agents such as thiotepa and cyclosphosphamide; alkyl sulfonates such as busulfan, improsulfan, and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, pomalidomide, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pemetrexed, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., nab-paclitaxel, paclitaxel and docetaxel; chlorambucil; gemcitabine; 6-thioguanine; mercaptopurine; methotrexate; platinum and platinum coordination complexes such as cisplatin, carboplatin and oxaliplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; proteasome inhibitors such as bortezomib, carfilzomib and ixazomib; topoisomerase inhibitors; difluoromethylornithine (DMFO); retinoic acid; esperamicins; capecitabine; anthracyclines; and pharmaceutically acceptable salts, acids or derivatives of any of the above. In some embodiments, the chemotherapeutic agent comprises a taxoid, platinum-based, or anthracycline-based chemotherapeutic agent. In some embodiments, the chemotherapeutic agent is selected from the group consisting of cisplatin, carboplatin, oxaliplatin, doxorubicin, paclitaxel, and docetaxel. In some embodiments, the chemotherapeutic agent is gemcitabine or nab-paclitaxel. In certain embodiments, combination therapy comprises a chemotherapy regimen that includes one or more chemotherapeutic agents. In one embodiment, combination therapy comprises a chemotherapeutic regimen comprising one or more of FOLFOX (folinic acid, fluorouracil, and oxaliplatin), FOLFIRI (e.g., folinic acid, fluorouracil, and irinotecan), a taxoid (e.g., docetaxel, paclitaxel, nab-paclitaxel,etc.), and/or gemcitabine.

In some embodiments, one or more of the additional therapeutic agents is a hormone therapy. Hormone therapies act to regulate or inhibit hormonal action on tumors Examples of hormone therapies include, but are not limited to: selective estrogen receptor degraders such as fulvestrant, giredestrantGDC-9545, SAR439859, RG6171, AZD9833, rintodestrant, ZN-c5, LSZ102, D-0502, LY3484356, SHR9549; selective estrogen receptor modulators such as tamoxifen, raloxifene, 4-hydroxytamoxifen, trioxifene, keoxifene, toremifene; aromatase inhibitors such as anastrozole, exemestane, letrozole and other aromatase inhibiting 4(5)-imidazoles; gonadotropin-releasing hormone agonists such as nafarelin, triptorelin, goserelin; gonadotropin-releasing hormone antagonists such as degarelix; antiandrogens such as abiraterone, enzalutamide, apalutamide, darolutamide, flutamide, nilutamide, bicalutamide, leuprolide; 5a-reductase inhibitors such as finasteride, dutasteride; and the like. In certain embodiments, combination therapy comprises a chemotherapy regimen that includes one or more chemotherapeutic agents. In certain embodiments, combination therapy comprises administration of a hormone or related hormonal agent. In one embodiment, combination therapy comprises administration of enzalutamide.

In some embodiments, one or more of the additional therapeutic agents is a radiopharmaceutical. A radiopharmaceutical is a form of internal radiation therapy in which a source of radiation (i.e., one or more radionuclide) is put inside a subject's body. Targeted radionuclides comprise a radionuclide associated (e.g., by covalent or ionic interactions) with a molecule (“a targeting agent”) that specifically binds to a target on a cell, typically a cancer cell or an immune cell. The targeting agent may be a small molecule, a saccharide (inclusive of oligosaccharides and polysaccharides), an antibody, a lipid, a protein, a peptide, a non-natural polymer, or an aptamer. In some embodiments, the targeting agent is a saccharide (inclusive of oligosaccharides and polysaccharides), a lipid, a protein, or a peptide and the target is a tumor-associated antigen (enriched but not specific to a cancer cell), a tumor-specific antigen (minimal to no expression in normal tissue), or a neo-antigen (an antigen specific to the genome of a cancer cell generated by non-synonymous mutations in the tumor cell genome). In some embodiments, the targeting agent is an antibody and the target is a tumor-associated antigen (i.e., an antigen enriched but not specific to a cancer cell), a tumor-specific antigen (i.e., an antigen with minimal to no expression in normal tissue), or a neo-antigen (i.e., an antigen specific to the genome of a cancer cell generated by non-synonymous mutations in the tumor cell genome). Non-limiting examples of targeted radionuclides include radionuclides attached to: somatostatin or peptide analogs thereof (e.g., 177Lu-Dotatate, etc.); prostate specific membrane antigen or peptide analogs thereof (e.g., 177Lu-PSMA-617, 225Ac-PSMA-617, 177Lu-PSMA-I&T, 177Lu-MIP-1095, etc.); a receptor's cognate ligand, peptide derived from the ligand, or variants thereof (e.g., 188Re-labeled VEGF₁₂₅₋₁₃₆ or variants thereof with higher affinity to VEGF receptor, etc.); and antibodies targeting tumor antigens (e.g., 131I-tositumomab, 90Y-ibritumomab tiuxetan, CAM-H2-I131 (Precirix NV), I131-omburtamab, etc.).

In some embodiments, one or more of the additional therapeutic agents is a targeted therapy. In one aspect, a targeted therapy may comprise a targeting agent and a drug. The drug may be a chemotherapeutic agent, a radionuclide, a hormone therapy, or another small molecule drug attached to a targeting agent. The targeting agent may be a small molecule, a saccharide (inclusive of oligosaccharides and polysaccharides), an antibody, a lipid, a protein, a peptide, a non-natural polymer, or an aptamer. In some embodiments, the targeting agent is a saccharide (inclusive of oligosaccharides and polysaccharides), a lipid, a protein, or a peptide and the target is a tumor-associated antigen (enriched but not specific to a cancer cell), a tumor-specific antigen (minimal to no expression in normal tissue), or a neo-antigen (an antigen specific to the genome of a cancer cell generated by non-synonymous mutations in the tumor cell genome). In some embodiments, the targeting agent is an antibody and the target is a tumor-associated antigen, a tumor-specific antigen, or a neo-antigen. In some embodiments, the targeted therapy is an antibody-drug conjugate comprising an antibody and a drug, wherein the antibody specifically binds to HER2, HER3, nectin-4, or Trop-2. Specific examples of a targeted therapy comprising an antibody and a drug include but are not limited to patritumab deruxtecan, sacituzumab govitecan-hziy, telisotuzumab vedotin, and trastuzumab deruxtecan. In other aspects, a targeted therapy may inhibit or interfere with a specific protein that helps a tumor grow and/or spread. Non-limiting examples of such targeted therapies include signal transduction inhibitors, RAS signaling inhibitors, inhibitors of oncogenic transcription factors, activators of oncogenic transcription factor repressors, angiogenesis inhibitors, immunotherapeutic agents, ATP-adenosine axis-targeting agents, AXL inhibitors, PARP inhibitors, PAK4 inhibitors, PI3K inhibitors, HIF-2α inhibitors, CD39 inhibitors, CD73 inhibitors, A2R antagonists, TIGIT antagonists, and PD-1 antagonists. Signal transduction inhibitors are described above, while other agents are described in further detail below.

In certain embodiments, the present disclosure contemplates the use of a compound of Formula (I) or (Ia) (e.g., as a reconstituted lyophilized formulation comprising the compound of Formula (I) or (Ia) described herein) in combination with other agents that modulate the level of adenosine (e.g., an ATP-adenosine axis-targeting agent). In some embodiments, the present disclosure contemplates combination with an ectonucleoside triphosphate diphosphohydrolase 1 (ENTPD1, also known as CD39 or Cluster of Differentiation 39) inhibitor. Exemplary anti-CD39 antibodies include ES002023, TTX-030, IPH-5201, SRF-617, CPI-006, and AB598. In some embodiments, the present disclosure contemplates combination with an adenosine receptor antagonist selected from the group consisting of etrumadenant, inupadenant, taminadenant, caffeine citrate, NUV-1182, TT-702, DZD-2269, INCB-106385, EVOEXS-21546, AZD-4635, imaradenant, RVU-330, ciforadenant, PBF-509, PBF-999, PBF-1129, and CS-3005. In some embodiments, the present disclosure contemplates the present disclosure contemplates the combination of the compound of Formula (I) or (Ia) described herein with an A2_{A}R antagonist, an A2_{B}R antagonist, or an antagonist of A2_{A}R and A2_{B}R. In some embodiments, the present disclosure contemplates combination with the adenosine receptor antagonists described in WO/2018/136700, WO 2018/204661, WO 2018/213377, or WO/2020/023846. In some embodiments, the at least one additional therapeutic agents comprise an adenosine pathway inhibitor that inhibits A_{2A}R, A_{2B}R, CD39, or combinations thereof. In some embodiments, the adenosine pathway inhibitor is etrumadenant, inupadenant, taminadenant, caffeine citrate, imaradenant, or ciforadenant. In one embodiment, the adenosine receptor antagonist (the adenosine pathway inhibitor) is etrumadenant (AB928).

In certain embodiments, the present disclosure contemplates the use of a compound of Formula (I) or (Ia) (e.g., as a reconstituted lyophilized formulation comprising a compound of Formula (I) or (Ia) described herein) in combination with inhibitors of phosphatidylinositol 3-kinases (PI3Ks), particularly the PI3Kγ isoform. In one embodiment, the PI3Kγ inhibitor is copanlisib, duvelisib, AT-104, ZX-101, tenalisib, eganelisib, SF-1126, AZD3458, or pictilisib. In another embodiment, the PI3K inhibitor is chosen from those described in WO/2020/247496.

In certain embodiments, the present disclosure contemplates the use of a compound of Formula (I) or (Ia) (e.g., as a reconstituted lyophilized formulation comprising a compound of Formula (I) or (Ia) described herein) with inhibitors of arginase, which has been shown to be either responsible for or to participate in inflammation-triggered immune dysfunction, tumor immune escape, immunosuppression and immunopathology of infectious disease. Suitable arginase inhibitors include CB-1158 and OAT-1746, as well as those described in WO 2019/173188 and WO/2020/102646.

In certain embodiments, the present disclosure contemplates the use of a compound of Formula (I) or (Ia) (e.g., as a reconstituted lyophilized formulation comprising a compound of Formula (I) or (Ia) described herein) with inhibitors of a hypoxia-inducible factor (HIF) transcription factor, particularly HIF-2a. In some embodiments, the compound of Formula (I) or (Ia) is combined with a HIF-2a inhibitor selected from the group consisting of belzutifan, ARO-HIF2, PT-2385, AB521, and those described in WO 2021113436 and WO 2021188769. In some embodiments, the at least one additional therapeutic agent comprises a HIF-2α inhibitor selected from the group consisting of belzutifan, ARO-HIF2, PT-2385, and AB521. In some embodiments, the HIF-2α inhibitor is AB521.

The present disclosure also contemplates the combination of a compound of Formula (I) or (Ia) (e.g., the reconstituted formulation described herein) with one or more RAS signaling inhibitors. Oncogenic mutations in the RAS family of genes, e.g., HRAS, KRAS, and NRAS, are associated with a variety of cancers. For example, mutations of G12C, G12D, G12V, G12A, G13D, Q61H, G13C and G12S, among others, in the KRAS family of genes have been observed in multiple tumor types. Direct and indirect inhibition strategies have been investigated for the inhibition of mutant RAS signaling. Indirect inhibitors target effectors other than RAS in the RAS signaling pathway, and include, but are not limited to, inhibitors of RAF, MEK, ERK, PI3K, PTEN, SOS (e.g., SOS1), mTORC1, SHP2 (PTPN11), and AKT. Non-limiting examples of indirect inhibitors under development include RMC-4630, RMC-5845, RMC-6291, RMC-6236, JAB-3068, JAB-3312, TNO155, RLY-1971, BI1701963. Direct inhibitors of RAS mutants have also been explored, and generally target the KRAS-GTP complex or the KRAS-GDP complex. Exemplary direct RAS inhibitors under development include, but are not limited to, sotorasib (AMG510), MRTX849, mRNA-5671 and ARS1620. In some embodiments, the one or more RAS signaling inhibitors are selected from the group consisting of RAF inhibitors, MEK inhibitors, ERK inhibitors, PI3K inhibitors, PTEN inhibitors, SOS1 inhibitors, mTORC1 inhibitors, SHP2 inhibitors, and AKT inhibitors. In other embodiments, the one or more RAS signaling inhibitors directly inhibit RAS mutants.

In some embodiments, this disclosure is directed to the combination of a compound of Formula (I) or (Ia) (e.g., as a reconstituted formulation described herein) with one or more inhibitors of anexelekto (i.e., AXL). There are a variety of AXL inhibitors under development that also inhibit other kinases in the TAM family (i.e., TYRO3, MERTK), as well as other receptor tyrosine kinases including MET, FLT3, RON and AURORA, among others. AXL specific inhibitors have also been developed, e.g., DS-1205, SGI-7079, TP-0903 (i.e., dubermatinib), BGB324 (i.e., bemcentinib) and DP3975; as well as anti-AXL antibodies such as ADCT-601; and antibody drug conjugates (ADCs) such as BA3011. Another strategy to inhibit AXL signaling involves targeting AXL's ligand, GAS6 (e.g., AVB-500). In some embodiments, the at least one additional therapeutic agent comprises a multi-tyrosine kinase inhibitor selected from gilteritinib, glesatinib, merestinib, cabozantinib, foretinib, rebastinib, sitravatinib, XL092, BMS777607, LY2801653, 549076, GSK1363089, and RXDX-106. In some embodiments, the at least one additional therapeutic agent according is an AXL inhibitor described in PCT/US2022/030227 or PCT/US2022/030230.

The present disclosure also contemplates the combination of a compound of Formula (I) or (Ia) (e.g., as a reconstituted formulation described herein) with one or more p21-activated kinase 4 (PAK4) inhibitors.

In some embodiments, one or more of the additional therapeutic agent is an epigenetic modulator. An epigenetic modulator alters an epigenetic mechanism controlling gene expression, and may be, for example, an inhibitor or activator of an epigenetic enzyme. Non-limiting examples of epigenetic modulators include DNA methyltransferase (DNMT) inhibitors, hypomethylating agents, and histone deacetylase (HDAC) inhibitors. In one or more embodiments, the compound of Formula (I) or (Ia) (e.g., the reconstituted formulation described herein) according to this disclosure are combined with DNA methyltransferase (DNMT) inhibitors or hypomethylating agents. Exemplary DNMT inhibitors include decitabine, zebularine and azacitadine. In one or more embodiments, combinations of the compound of Formula (I) or (Ia) (e.g., the reconstituted formulation described herein) according to this disclosure with a histone deacetylase (HDAC) inhibitor is also contemplated. Exemplary HDAC inhibitors include vorinostat, givinostat, abexinostat, panobinostat, belinostat, and trichostatin A.

In some embodiments, one or more of the additional therapeutic agent is an inhibitor of an oncogenic transcription factor or an activator of an oncogenic transcription factor repressor. Suitable agents may act at the expression level (e.g., RNAi, siRNA, etc.), through physical degradation, at the protein/protein level, at the protein/DNA level, or by binding in an activation/inhibition pocket. Non-limiting examples include inhibitors of one or more subunit of the MLL complex (e.g., HDAC, DOT1L, BRD4, Menin, LEDGF, WDR5, KDM4C (JMJD2C) and PRMT1), inhibitors of hypoxia-inducible factor (HIF) transcription factor, and the like.

In some embodiments, one or more of the additional therapeutic agents is (i) an agent that inhibits the enzyme poly (ADP-ribose) polymerase (e.g., olaparib, niraparib and rucaparib, etc.); (ii) an inhibitor of the Bcl-2 family of proteins (e.g., venetoclax, navitoclax, etc.); (iii) an inhibitor of MCL-1; (iv) an inhibitor of the CD47-SIRPα pathway (e.g., the anti-CD47 antibody, magrolimab, etc.); or (v) an isocitrate dehydrogenase (IDH) inhibitor, e.g., IDH-1 or IDH-2 inhibitor (e.g., ivosidenib, enasidenib, etc.).

In some embodiments, one or more of the additional therapeutic agents is an immunotherapeutic agent. Immunotherapeutic agents treat a disease by stimulating or suppressing the immune system. Immunotherapeutic agents useful in the treatment of cancers typically elicit or amplify an immune response to cancer cells. Non-limiting examples of suitable immunotherapeutic agents include: immunomodulators; cellular immunotherapies; vaccines; gene therapies; ATP-adenosine axis-targeting agents; immune checkpoint modulators; and certain signal transduction inhibitors. ATP-adenosine axis-targeting agents and signal transduction inhibitors are described above. Immunomodulators, cellular immunotherapies, vaccines, gene therapies, and immune checkpoint modulators are described further below.

In some embodiments, the one or more additional therapeutic agent is an immunotherapeutic agent, more specifically a cytokine or chemokine, such as, IL1, IL2, IL12, IL18, ELC/CCL19, SLC/CCL21, MCP-1, IL-4, IL-18, TNF, IL-15, MDC, IFNa/b, M-CSF, IL-3, GM-CSF, IL-13, and anti-IL-10; bacterial lipopolysaccharides (LPS); an organic or inorganic adjuvant that activates antigen-presenting cells and promotes the presentation of antigen epitopes on major histocompatibility complex molecules agonists including, but not limited to Toll-like receptor (TLR) agonists, antagonists of the mevalonate pathway, agonists of STING; indoleamine 2,3-dioxygenase 1 (IDO1) inhibitors; and immune-stimulatory oligonucleotides, as well as other T cell adjuvants.

In some embodiments, one or more of the additional therapeutic agents is an immunotherapeutic agent, more specifically a cellular therapy. Cellular therapies are a form of treatment in which viable cells are administered to a subject. In certain embodiments, one or more of the additional therapeutic agents is a cellular immunotherapy that activates or suppresses the immune system. Cellular immunotherapies useful in the treatment of cancers typically elicit or amplify an immune response. The cells can be autologous or allogenic immune cells (e.g., monocytes, macrophages, dendritic cells, NK cells, T cells, etc.) collected from one or more subject. Alternatively, the cells can be “(re)programmed” allogenic immune cells produced from immune precursor cells (e.g., lymphoid progenitor cells, myeloid progenitor cells, common dendritic cell precursor cells, stem cells, induced pluripotent stem cells, etc.). In some embodiments, such cells may be an expanded subset of cells with distinct effector functions and/or maturation markers (e.g., adaptive memory NK cells, tumor infiltrating lymphocytes, immature dendritic cells, monocyte-derived dendritic cells, plasmacytoid dendritic cells, conventional dendritic cells (sometimes referred to as classical dendritic cells), M1 macrophages, M2 macrophages, etc.), may be genetically modified to target the cells to a specific antigen and/or enhance the cells' anti-tumor effects (e.g., engineered T cell receptor (TCR) cellular therapies, chimeric antigen receptor (CAR) cellular therapies, lymph node homing of antigen-loaded dendritic cells, etc.), may be engineered to express or have increased expression of a tumor-associated antigen, or may be any combination thereof. Non-limiting types of cellular therapies include CAR-T cell therapy, CAR-NK cell therapy, TCR therapy, and dendritic cell vaccines. Exemplary cellular immunotherapies include sipuleucel-T, tisagenlecleucel, lisocabtagene maraleucel, idecabtagene vicleucel, brexucabtagene autoleucel, and axicabtagene ciloleucel, as well as CTX110, JCAR015, JCAR017, MB-CART19.1,MB-CART20.1, MB-CART2019.1, UniCAR02-T-CD123, BMCA-CAR-T, JNJ-68284528, BNT211, and NK-92/5.28.z.

In some embodiments, one or more of the additional therapeutic agents is an immunotherapeutic agent, more specifically a gene therapy. Gene therapies comprise recombinant nucleic acids administered to a subject or to a subject's cells ex vivo in order to modify the expression of an endogenous gene or to result in heterologous expression of a protein (e.g., small interfering RNA (siRNA) agents, double-stranded RNA (dsRNA) agents, micro RNA (miRNA) agents, viral or bacterial gene delivery, etc.), as well as gene editing therapies that may or may not comprise a nucleic acid component (e.g., meganucleases, zinc finger nucleases, TAL nucleases, CRISPR/Cas nucleases, etc.), oncolytic viruses, and the like. Non-limiting examples of gene therapies that may be useful in cancer treatment include Gendicine® (rAd-p53), Oncorine® (rAD5-H101), talimogene laherparepvec, Mx-dnG1, ARO-HIF2 (Arrowhead), quaratusugene ozeplasmid (Immunogene), CTX110 (CRISPR Therapeutics), CTX120 (CRISPR Therapeutics), and CTX130 (CRISPR Therapeutics).

In some embodiments, one or more of the additional therapeutic agent is an immunotherapeutic agent, more specifically an agent that modulates an immune checkpoint. Immune checkpoints are a set of inhibitory and stimulatory pathways that directly affect the function of immune cells (e.g., B cells, T cells, NK cells, etc.). Immune checkpoints engage when proteins on the surface of immune cells recognize and bind to their cognate ligands. The present disclosure contemplates the use of a compound of Formula (I) or (Ia) (e.g., as a reconstituted lyophilized formulation comprising a compound of Formula (I) or (Ia) described herein) in combination with agonists of stimulatory or co-stimulatory pathways and/or antagonists of inhibitory pathways. Agonists of stimulatory or co-stimulatory pathways and antagonists of inhibitory pathways may have utility as agents to overcome distinct immune suppressive pathways within the tumor microenvironment, inhibit T regulatory cells, reverse/prevent T cell anergy or exhaustion, trigger innate immune activation and/or inflammation at tumor sites, or combinations thereof.

In some embodiments, one or more of the additional therapeutic agents is an immune checkpoint inhibitor. As used herein, the term “immune checkpoint inhibitor” refers to an antagonist of an inhibitory or co-inhibitory immune checkpoint. The terms “immune checkpoint inhibitor”, “checkpoint inhibitor” and “CPI” may be used herein interchangeably. Immune checkpoint inhibitors may antagonize an inhibitory or co-inhibitory immune checkpoint by interfering with receptor -ligand binding and/or altering receptor signaling. Examples of immune checkpoints (ligands and receptors), some of which are selectively upregulated in various types of cancer cells, that can be antagonized include PD-1 (programmed cell death protein 1); PD-L1 (PD1 ligand); BTLA (B and T lymphocyte attenuator); CTLA-4 (cytotoxic T-lymphocyte associated antigen 4); TIM-3 (T cell immunoglobulin and mucin domain containing protein 3); LAG-3 (lymphocyte activation gene 3); TIGIT (T cell immunoreceptor with Ig and ITIM domains); CD276 (B7-H3), PD-L2, Galectin 9, CEACAM-1, BTLA, CD69, Galectin-1, CD113, GPR56, VISTA, 2B4, CD48, GARP, PD1H, LAIR1, TIM-1, and TIM-4, and Killer Inhibitory Receptors, which can be divided into two classes based on their structural features: i) killer cell immunoglobulin-like receptors (KIRs), and ii) C-type lectin receptors (members of the type II transmembrane receptor family). Also contemplated are other less well-defined immune checkpoints that have been described in the literature, including both receptors (e.g., the 2B4 (also known as CD244) receptor) and ligands (e.g., certain B7 family inhibitory ligands such B7-H3 (also known as CD276) and B7-H4 (also known as B7-S1, B7x and VCTN1)). In some embodiments, the at least one additional therapeutic agent comprises one or more immune checkpoint inhibitors that target PD-1, PD-L1, TIGIT, CTLA-4, TIM-3, LAG-3, a B7 family member, or any combination thereof. In some embodiments, the at least one additional therapeutic agents comprise an immune checkpoint inhibitor that targets PD-1 or PD-L1. In some embodiments, the at least one additional therapeutic agent comprises an immune checkpoint inhibitor that targets TIGIT.

In some embodiments, an immune checkpoint inhibitor is a CTLA-4 antagonist. In further embodiments, the CTLA-4 antagonist can be an antagonistic CTLA-4 antibody. Suitable antagonistic CTLA-4 antibodies include, for example, monospecific antibodies such as ipilimumab or tremelimumab, as well as bispecific antibodies such as MEDI5752 and KN046.

In some embodiments, an immune checkpoint inhibitor is a PD-1 antagonist. In further embodiments, the PD-1 antagonist can be an antagonistic PD-1 antibody, small molecule or peptide. Suitable antagonistic PD-1 antibodies include, for example, monospecific antibodies such as balstilimab, budigalimab, camrelizumab, cosibelimab, dostarlimab, cemiplimab, ezabenlimab, MEDI-0680 (AMP-514; WO2012/145493), nivolumab, pembrolizumab, pidilizumab, pimivalimab, retifanlimab, sasanlimab, spartalizumab, sintilimab, tislelizumab, toripalimab, and zimberelimab; as well as bi-specific antibodies such as LY3434172. In still further embodiments, the PD-1 antagonist can be a recombinant protein composed of the extracellular domain of PD-L2 (B7-DC) fused to the Fc portion of IgG1 (AMP-224).

In some embodiments, the immune checkpoint inhibitor is nivolumab, pembrolizumab, avelumab, atezolizumab, durvalumab, cemiplimab or zimberelimab. In certain embodiments, an immune checkpoint inhibitor is zimberelimab.

In some embodiments, an immune checkpoint inhibitor is a PD-L1 antagonist. In further embodiments, the PD-L1 antagonist can be an antagonistic PD-L1 antibody. Suitable antagonistic PD-L1 antibodies include, for example, monospecific antibodies such as avelumab, atezolizumab, durvalumab, BMS-936559, and envafolimab as well as bi-specific antibodies such as LY3434172 and KN046.

In some embodiments, an immune checkpoint inhibitor is a TIGIT antagonist. In further embodiments, the TIGIT antagonist can be an antagonistic TIGIT antibody. Suitable antagonistic anti-TIGIT antibodies include monospecific antibodies such as AGEN1327, AB308 (WO2021247591), BMS 986207, COM902, domvanalimab, EOS-448, etigilimab, IBI-929, JS006, M6223, ociperlimab, SEA-TGT, tiragolumab, and vibostolimab; as well as bi-specific antibodies such as AGEN1777 and AZD2936. In certain embodiments, an immune checkpoint inhibitor is an antagonistic anti-TIGIT antibody disclosed in W02017152088 or W02021247591. In some embodiments, the immune checkpoint inhibitor is domvanalimab, etigilimab, ociperlimab, AB308, tiragolumab, or vibostolimab. In certain embodiments, an immune checkpoint inhibitor is domvanalimab or AB308.

In another aspect, the immune checkpoint inhibitor is a LAG-3 antagonist, such as an antagonistic LAG-3 antibody. Suitable LAG-3 antibodies include, for example, BMS-986016 (WO10/19570, WO14/08218), or IMP-731 or IMP-321 (WO08/132601, WO09/44273).

In certain embodiments, an immune checkpoint inhibitor is a B7-H3 antagonist. In further embodiments, the B7-H3 antagonist is an antagonistic B7-H3 antibody. Suitable antagonist B7-H3 antibodies include, for example, MGA271 (OW11/109400), omburtumab, enoblituzumab, DS-7300a, ABBV-155, and SHR-A1811.

In some embodiments, one or more of the additional therapeutic agents activates a stimulatory or co-stimulatory immune checkpoint. Examples of stimulatory or co-stimulatory immune checkpoints (ligands and receptors) include B7-1, B7-2, CD28, 4-1BB (CD137), 4-1BBL, ICOS, ICOS-L, OX40, OX40L, GITR, GITRL, CD70, CD27, CD40, DR3 and CD2.

In some embodiments, an agent that activates a stimulatory or co-stimulatory immune checkpoint is a CD137 (4-1BB) agonist. In further embodiments, the CD137 agonist can be an agonistic CD137 antibody. Suitable CD137 antibodies include, for example, urelumab and PF-05082566 (WO12/32433). In some embodiments, an agent that activates a stimulatory or co-stimulatory immune checkpoint is a GITR agonist. In further embodiments, the GITR agonist can be an agonistic GITR antibody. Suitable GITR antibodies include, for example, BMS-986153, BMS-986156, TRX-518 (WO06/105021, WO09/009116) and MK-4166 (WO11/028683). In some embodiments, an agent that activates a stimulatory or co-stimulatory immune checkpoint is an OX40 agonist. In further embodiments, the OX40 agonist can be an agonistic OX40 antibody. Suitable OX40 antibodies include, for example, MEDI-6383, MEDI-6469, MEDI-0562, PF-04518600, GSK3174998, BMS-986178, and MOXR0916. In some embodiments, an agent that activates a stimulatory or co-stimulatory immune checkpoint is a CD40 agonist. In further embodiments, the CD40 agonist can be an agonistic CD40 antibody. In some embodiments, an agent that activates a stimulatory or co-stimulatory immune checkpoint is a CD27 agonist. In further embodiments, the CD27 agonist can be an agonistic CD27 antibody. Suitable CD27 antibodies include, for example, varlilumab.

In some embodiments, one or more of the additional therapeutic agents is an immunotherapeutic agent, more specifically a signal transduction inhibitor. Intracellular signaling molecules that influence immune cell functions may also be suitable targets for improving antitumor immunity For example, one or more of the additional therapeutic agents may be an inhibitor of hematopoietic progenitor kinase 1 (HPK1). HPK1 is serine/threonine kinase that functions as a negative regulator of activation signals generated by the T cell antigen receptor.

In some embodiments, one or more of the additional therapeutic agents is an agent that inhibits or depletes immune-suppressive immune cells. For example, to inhibit or deplete immunosuppressive macrophages or monocytes the agent may be CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264).

In some embodiments, each additional therapeutic agent can independently be a chemotherapeutic agent, a radiopharmaceutical, a hormone therapy, an epigenetic modulator, a targeted agent, an immunotherapeutic agent, a cellular therapy, or a gene therapy. For example, in one embodiment, the present disclosure contemplates the use of the formulations described herein in combination with one or more chemotherapeutic agent and optionally one or more additional therapeutic agents, wherein each additional therapeutic agent is independently a radiopharmaceutical, a hormone therapy, a targeted agent, an immunotherapeutic agent, a cellular therapy, or a gene therapy. In another embodiment, the present disclosure contemplates the use of the formulations described herein in combination with one or more chemotherapeutic agents and optionally one or more additional therapeutic agents, wherein each additional therapeutic agent is independently a targeted agent, an immunotherapeutic agent, or a cellular therapy. In another embodiment, the present disclosure contemplates the use of the formulations described herein in combination with one or more immunotherapeutic agents and optionally one or more additional therapeutic agent, wherein each additional therapeutic agent is independently a radiopharmaceutical, a hormone therapy, a targeted agent, a chemotherapeutic agent, a cellular therapy, or a gene therapy. In another embodiment, the present disclosure contemplates the use of the formulations described herein in combination with one or more immunotherapeutic agents and optionally one or more additional therapeutic agents, wherein each additional therapeutic agent is independently a chemotherapeutic agent, a targeted agent, or a cellular therapy. In another embodiment, the present disclosure contemplates the use of the formulations described herein in combination with one or more immune checkpoint inhibitors and/or one or more ATP-adenosine axis-targeting agents, and optionally one or more additional therapeutic agents, wherein each additional therapeutic agent is independently a chemotherapeutic agent, a targeted agent, an immunotherapeutic agent, or a cellular therapy. In further embodiments of the above (a) the targeted agent can be a PI3K inhibitor, an arginase inhibitor, a HIF2a inhibitor, an AXL inhibitor, or a PAK4 inhibitor; (b) the immunotherapeutic agent is an ATP-adenosine axis-targeting agent or an immune checkpoint inhibitor; (c) the ATP-adenosine axis-targeting agent is an A2 A R and/or A2 B R antagonist or a CD39 inhibitor; (d) the ATP-adenosine axis-targeting agent is etrumadenant; (e) the immunotherapeutic agent is an anti-PD-1 antagonist antibody or an anti-TIGIT antagonist antibody; (f) the immunotherapeutic agent is zimberelimab, domvanalimab, or AB308; or (g) any combination thereof. In still further embodiments of the above, the present disclosure contemplates the use of the formulations described herein in combination with domvanalimab, etrumadenant, zimberelimab, AB308, AB521, AB610, or any combination thereof.

In some embodiments, methods described herein comprise administering at least one additional therapeutic agent to the subject. In some embodiments, the at least one additional therapeutic agent comprises one or more agents selected from the groups consisting of chemotherapeutic agents, immune checkpoint inhibitors, inhibitors of HIF-2α, adenosine pathway inhibitors, radiation therapy, and multi-tyrosine kinase inhibitors

Some embodiments provide for a method of treating cancer, said method comprising administering a compound of Formula (Ia) or a pharmaceutically acceptable salt thereof, in combination with an anti-PD-1 antagonistic antibody and an anti-TIGIT antagonistic antibody, wherein compound of Formula (Ia) is administered in an amount ranging from 50 mg to 300 mg every two to three weeks, the anti-PD-1 antagonistic antibody is administered in an amount ranging from 300 mg to 600 mg every two to five weeks, and the anti-TIGIT antagonistic antibody is administered in an amount ranging from 1200 mg to about 1600 mg every two to four weeks.

In some embodiments, the compound of Formula (Ia) is administered in an amount of about 300 mg every three weeks. In some embodiments, the anti-PD-1 antagonistic antibody is administered in an amount of about 300 mg every three weeks. In some embodiments, the anti-TIGIT antagonistic antibody is administered in an amount ranging from about 1200 mg to about 1600 mg every three weeks. In some embodiments, the anti-PD-1 antagonistic antibody is zimberelimab. In some embodiments, the anti-TIGIT antagonistic antibody is domvanalimab or AB308. In some embodiments, the compound of Formula (Ia) is a reconstituted aqueous solution as described herein. In some embodiments, the compound of Formula (Ia), the anti-PD-1 antagonistic antibody, and the anti-TIGIT antagonistic antibody are administered intravenously on a three week cycle.

In some embodiments, methods describe herein further comprise one or more additional therapeutic agents. In some embodiments, the one or more additional therapeutic agents comprise chemotherapy.

Some embodiments provide for a method of treating cancer, said method comprising administering a compound of Formula (Ia) or a pharmaceutically acceptable salt thereof, in combination with chemotherapy, wherein compound of Formula (Ia) is administered in an amount ranging from 50 mg to 300 mg every two to three weeks.

Examples of therapeutic agents useful in combination therapy for immune- and inflammatory-related diseases, disorders or conditions include, but are not limited to, the following: non-steroidal anti-inflammatory drugs (NSAIDs), steroids such as prednisolone, prednisone, methylprednisolone, betamethasone, dexamethasone, or hydrocortisone or cytokine suppressive anti-inflammatory drug(s) (CSAIDs).

### Dosing

The lyophilized formulations comprising a compound of Formula (I) or (Ia) may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered.

In general, dosing parameters dictate that the dosage amount be less than an amount that could be irreversibly toxic to the subject (the maximum tolerated dose (MTD)) and not less than an amount required to produce a measurable effect on the subject. Such amounts are determined by, for example, the pharmacokinetic and pharmacodynamic parameters associated with ADME, taking into consideration the route of administration and other factors.

In certain embodiments, the compound of Formula (I) or (Ia) (e.g., as a reconstituted formulation described herein) may be administered (e.g., parenterally) at dosage levels of about 0.01 mg/kg to about 50 mg/kg, or about 1 mg/kg to about 25 mg/kg, of subject body weight per day, one or more times a day, to obtain the desired therapeutic effect.

In some embodiments, the lyophilized formulation contains from 1 to 500 milligrams of the active ingredient (i.e., a compound of Formula (I) or (Ia)), particularly 20, 25, 30, 100, 150, 200, 225, 250, 275, 300, 325, 400, and 500 milligrams of the active ingredient. In some embodiments, the compound of Formula (I) or (Ia) is provided in an amount of 25, 50, 75, 100, 150, 200, 250, 300, or 350 milligrams. In some embodiments, the compound of Formula (I) or (Ia) is provided in an amount of 25 milligrams to 350 milligrams. In some embodiments, the compound of Formula (I) or (Ia) is provided in an amount of 25 milligrams to 300 milligrams In some embodiments, the compound of Formula (I) or (Ia) is provided in an amount of 25 milligrams to 250 milligrams. In some embodiments, the compound of Formula (I) or (Ia) is provided in an amount of 25 milligrams to 200 milligrams. In some embodiments, the compound of Formula (I) or (Ia) is provided in an amount of 25 milligrams to 150 milligrams. In some embodiments, the compound is provided in an amount of 25 milligrams to 120 milligrams. In some embodiments, the compound is provided in an amount of 25 milligrams to 110 milligrams. In some embodiments, the compound is provided in an amount of 25 milligrams to 100 milligrams. In some embodiments, the compound is provided in an amount of 25 milligrams to 75 milligrams. In some embodiments, the compound is provided in an amount of 25 milligrams to 50 milligrams. In some embodiments, the compound is provided in an amount of 75 milligrams to 100 milligrams. In some embodiments, the compound is provided in an amount of 50 milligrams to 200 milligrams. In some embodiments, the compound is provided in an amount of 75 milligrams to 150 milligrams. In some embodiments, the compound is provided in an amount of 75 milligrams to 125 milligrams. In some embodiments, the compound is provided in an amount of 75 milligrams to 110 milligrams. In some embodiments, the compound is provided in an amount of 90 milligrams to 110 milligrams.

In some embodiments, the compound of Formula (I) or (Ia) (e.g., the reconstituted formulation described herein) may be administered (e.g., parenterally) on a monthly, weekly or daily basis. In some embodiments, the compound of Formula (I) or (Ia) may be administered at least once a month, such as twice a month, three times a month, four times a month, once a week, or daily. In some embodiments, the compound of Formula (I) or (Ia) may be administered once every week, once every 2 weeks, once every 3 weeks, once every 4 weeks, once every 5 weeks, or once every 6 weeks. In some embodiments the compound of Formula (I) or (Ia) may be administered (e.g., parenterally) 1, 2, 3, or 4 times a month. In some embodiments, the compound of Formula (I) or (Ia) may be administered (e.g., parenterally) once a week, or once every two weeks, or once every three weeks.

In some embodiments, the lyophilized formulation according to this disclosure is reconstituted to form a reconstituted solution and is administered to a subject parenterally as a loading dose. The loading dose can be administered in order to achieve a suitable plasma concentration in the subject. Once a suitable plasma concentration has been achieved, subsequent doses of the compound of Formula (I) or (Ia) may be administered by other means (e.g., orally). In some embodiments, the loading dose is between about 20 mg to about 500 mg, such as about 20 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, 350 mg, about 375 mg, about 400 mg, about 425 mg, about 450 mg, about 475 mg, or about 500 mg. In some embodiments, the loading dose is between 20 mg to 500 mg, such as 20 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, or 500 mg.

In some embodiments, the compound of Formula (I) or (Ia) (e.g., the reconstituted formulation described herein) may be administered (e.g., parenterally) at a dose of about 10 mg to 225 mg once every two weeks (Q2W), such as, for example, 10 mg, 15 mg, 20 mg, 25 mg, 30 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, or 250 mg once every two weeks. In some embodiments, the compound of Formula (I) or (Ia) (e.g., the reconstituted formulation described herein) may be administered (e.g., parenterally) at a dose of about 10 mg to about 250 mg once every two weeks (Q2W), such as, for example, 10 mg, 15 mg, 20 mg, 25 mg, 30 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, or 150 mg once every two weeks. In one embodiment, the compound of Formula (I) or (Ia) (e.g., the reconstituted formulation described herein) is administered (e.g., parenterally) at a dose of 25 mg once every two weeks. In another embodiment, the compound of Formula (I) or (Ia) (e.g., the reconstituted formulation described herein) is administered (e.g., parenterally) at a dose of 50 mg once every two weeks. In one embodiment, the compound of Formula (I) or (Ia) (e.g., the reconstituted formulation described herein) is administered (e.g., parenterally) at a dose of 75 mg once every two weeks. In yet another embodiment, the compound of Formula (I) or (Ia) (e.g., the reconstituted formulation described herein) may be administered (e.g., parenterally) at a dose of 100 mg once every two weeks. In another embodiment, the compound of Formula (I) or (Ia) (e.g., the reconstituted formulation described herein) may be administered (e.g., parenterally) at a dose of 125 mg once every two weeks. In yet another embodiment, the compound of Formula (I) or (Ia) (e.g., the reconstituted formulation described herein) may be administered (e.g., parenterally) at a dose of 150 mg once every two weeks. In yet another embodiment, the compound of Formula (I) or (Ia) (e.g., the reconstituted formulation described herein) may be administered (e.g., parenterally) at a dose of 175 mg once every two weeks. In yet another embodiment, the compound of Formula (I) or (Ia) (e.g., the reconstituted formulation described herein) may be administered (e.g., parenterally) at a dose of 200 mg once every two weeks. In yet another embodiment, the compound of Formula (I) or (Ia) (e.g., the reconstituted formulation described herein) may be administered (e.g., parenterally) at a dose of 225 mg once every two weeks.

In some embodiments, the compound of Formula (I) or (Ia) (e.g., as a reconstituted formulation described herein) may be administered (e.g., parenterally) at a dose of about 10 mg to about 350 mg once every three weeks (Q3W), such as, for example, 10 mg, 15 mg, 20 mg, 25 mg, 30 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 250, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, or 350 mg once every three weeks. In some embodiments, the compound of Formula (I) or (Ia) (e.g., as a reconstituted formulation described herein) may be administered (e.g., parenterally) at a dose of about 10 mg to 250 mg once every three weeks (Q3W), such as, for example, 10 mg, 15 mg, 20 mg, 25 mg, 30 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, or 150 mg once every three weeks. In one embodiment, the compound of Formula (I) or (Ia) (e.g., the reconstituted formulation described herein) is administered (e.g., parenterally) at a dose of 25 mg once every three weeks. In another embodiment, the compound of Formula (I) or (Ia) (e.g., the reconstituted formulation described herein) is administered (e.g., parenterally) at a dose of 50 mg once every three weeks. In one embodiment, the compound of Formula (I) or (Ia) (e.g., the reconstituted formulation described herein) is administered (e.g., parenterally) at a dose of 75 mg once every three weeks. In yet another embodiment, the compound of Formula (I) or (Ia) (e.g., the reconstituted formulation described herein) may be administered (e.g., parenterally) at a dose of 100 mg once every three weeks. In another embodiment, the compound of Formula (I) or (Ia) (e.g., the reconstituted formulation described herein) may be administered (e.g., parenterally) at a dose of 125 mg once every three weeks. In yet another embodiment, the compound of Formula (I) or (Ia) (e.g., the reconstituted formulation described herein) may be administered (e.g., parenterally) at a dose of 150 mg once every three weeks. In yet another embodiment, the compound of Formula (I) or (Ia) (e.g., the reconstituted formulation described herein) may be administered (e.g., parenterally) at a dose of 175 mg once every three weeks. In yet another embodiment, the compound of Formula (I) or (Ia) (e.g., the reconstituted formulation described herein) may be administered (e.g., parenterally) at a dose of 200 mg once every three weeks. In yet another embodiment, the compound of Formula (I) or (Ia) (e.g., the reconstituted formulation described herein) may be administered (e.g., parenterally) at a dose of 225 mg once every three weeks. In yet another embodiment, the compound of Formula (I) or (Ia) (e.g., as a reconstituted formulation described herein) may be administered (e.g., parenterally) at a dose of 300 mg once every three weeks.

In some embodiments, the compound of Formula (I) or (Ia) (e.g., as a reconstituted formulation described herein) may be administered (e.g., parenterally) at a dose of about 10 mg to about 325 mg once every four weeks (Q4W), such as, for example, 10 mg, 15 mg, 20 mg, 25 mg, 30 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 250, 255 mg, 260 mg, 265 mg, 270 mg, 275 mg, 280 mg, 285 mg, 290 mg, 295 mg, 300 mg, 305 mg, 310 mg, 315 mg, 320 mg, or 325 mg once every four weeks. In some embodiments, the compound of Formula (I) or (Ia) (e.g., the reconstituted formulation described herein) may be administered (e.g., parenterally) at a dose of about 10 mg to 250 mg once every four weeks (Q4W), such as, for example, 10 mg, 15 mg, 20 mg, 25 mg, 30 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, or 150 mg once every four weeks. In one embodiment, the compound of Formula (I) or (Ia) (e.g., the reconstituted formulation described herein) is administered (e.g., parenterally) at a dose of 25 mg once every four weeks. In another embodiment, the compound of Formula (I) or (Ia) (e.g., the reconstituted formulation described herein) is administered (e.g., parenterally) at a dose of 50 mg once every four weeks. In one embodiment, the compound of Formula (I) or (Ia) (e.g., the reconstituted formulation described herein) is administered (e.g., parenterally) at a dose of 75 mg once every four weeks. In yet another embodiment, the compound of Formula (I) or (Ia) (e.g., the reconstituted formulation described herein) may be administered (e.g., parenterally) at a dose of 100 mg once every four weeks. In another embodiment, the compound of Formula (I) or (Ia) (e.g., the reconstituted formulation described herein) may be administered (e.g., parenterally) at a dose of 125 mg once every four weeks. In yet another embodiment, the compound of Formula (I) or (Ia) (e.g., the reconstituted formulation described herein) may be administered (e.g., parenterally) at a dose of 150 mg once every four weeks. In yet another embodiment, the compound of Formula (I) or (Ia) (e.g., the reconstituted formulation described herein) may be administered (e.g., parenterally) at a dose of 175 mg once every four weeks. In yet another embodiment, the compound of Formula (I) or (Ia) (e.g., the reconstituted formulation described herein) may be administered (e.g., parenterally) at a dose of 200 mg once every four weeks. In yet another embodiment, the compound of Formula (I) or (Ia) (e.g., the reconstituted formulation described herein) may be administered (e.g., parenterally) at a dose of 225 mg once every four weeks. In yet another embodiment, the compound of Formula (I) or (Ia) (e.g., as a reconstituted formulation described herein) may be administered (e.g., parenterally) at a dose of 300 mg once every four weeks.

In certain embodiments, the dosage of the compound of Formula (I) or (Ia) is contained in a “unit dosage form”. The phrase “unit dosage form” refers to physically discrete units, each unit containing a predetermined amount of the compound of Formula (I) or (Ia), either alone or in combination with one or more additional agents, sufficient to produce the desired effect. The unit dosage form may be provided as a pre-lyophilized composition, a lyophilized formulation or a reconstituted lyophilized formulation.

In some embodiments, provided herein is a single dosage form of a compound having Formula (Ia), wherein the compound is present in an amount of between about 20 mg to about 300 mg.

In one embodiment, provided herein is a single dosage form of a compound having Formula (Ia), wherein the compound is present in an amount of 25 mg.

In one embodiment, provided herein is a single dosage form of a compound having Formula (Ia), wherein the compound is present in an amount of 50 mg.

In one embodiment, provided herein is a single dosage form of a compound having Formula (Ia), wherein the compound is present in an amount of 75 mg.

In one embodiment, provided herein is a single dosage form of a compound having Formula (Ia), wherein the compound is present in an amount of 100 mg, 125 mg, 150 mg, 175 mg, 200 mg, 225 mg, 250 mg, 275 mg, 300 mg, or 325 mg.

In another embodiment, the lyophilized formulation described herein comprises an amount of a compound of Formula (I), or Formula (Ia), in an amount sufficient to achieve a desirable administration dose upon reconstitution of the lyophilized formulation. In some embodiments, the lyophilized formulation contains an amount of the compound of Formula (I), or Formula (Ia), in a range including the desired administration dose to the desired administration dose plus about 10% of the compound of Formula (I) or Formula (Ia). The larger amount of the compound may be provided to account for residual drug volumes remaining in the vial when the reconstituted solution is drawn from the vial for administration and/or dilution.

In some embodiments, the compound is present in the lyophilized formulation in an amount of about 25 mg to about 330 mg. In some embodiments, the compound is present in an amount of between about 25 mg to about 27.5 mg. In some embodiments, the compound is present in an amount of between about 50 mg to about 55 mg. In some embodiments, the compound is present in an amount of between about 75 mg to about 82.5 mg. t In some embodiments, he compound is present in an amount of about 100 mg to about 110 mg. In some embodiments, the compound is present in an amount of about 200 mg to about 325 mg.

In one embodiment, the compound is present in the lyophilized formulation in an amount of between about 25.0 mg to about 27.5 mg, such as 25.0 mg, 25.1 mg, 25.2 mg, 25.3 mg, 25.4 mg, 25.5 mg, 25.6 mg, 25.7 mg, 25.8 mg, 25.9 mg, 26.0 mg, 26.1 mg, 26.2 mg, 26.3 mg, 26.4 mg, 26.5 mg, 26.6 mg, 26.7 mg, 26.8 mg, 26.9 mg, 27.0 mg, 27.1 mg, 27.2 mg, 27.3 mg, 27.4 mg, or 27.5 mg. In one embodiment, the compound is present in the lyophilized formulation in an amount of between about 25.0 mg to about 27.5 mg, such as about 25.0 mg, about 25.1 mg, about 25.2 mg, about 25.3 mg, about 25.4 mg, about 25.5 mg, about 25.6 mg, about 25.7 mg, about 25.8 mg, about 25.9 mg, about 26.0 mg, about 26.1 mg, about 26.2 mg, about 26.3 mg, about 26.4 mg, about 26.5 mg, about 26.6 mg, about 26.7 mg, about 26.8 mg, about 26.9 mg, about 27.0 mg, about 27.1 mg, about 27.2 mg, about 27.3 mg, about 27.4 mg, or about 27.5 mg.

In one embodiment, the compound is present in the lyophilized formulation in an amount of between about 50.0 mg to about 55.0 mg, such as 50.0 mg, 50.5 mg, 51.0 mg, 51.5 mg, 52.0 mg, 52.5 mg, 53.0 mg, 53.5 mg, 54.0 mg, 54.5, or 55.0 mg. In one embodiment, the compound is present in the lyophilized formulation in an amount of between about 50.0 mg to about 55.0 mg, such as about 50.0 mg, about 50.5 mg, about 51.0 mg, about 51.5 mg, about 52.0 mg, about 52.5 mg, about 53.0 mg, about 53.5 mg, about 54.0 mg, about 54.5, or about 55.0 mg.

In one embodiment, the compound is present in the lyophilized formulation in an amount of between about 75.0 mg to about 82.5 mg, such as 75.0 mg, 75.5 mg, 76.0 mg, 76.5 mg, 77.0 mg, 77.5 mg, 78.0 mg, 78.5 mg, 79.0 mg, 79.5 mg, 80.0 mg, 80.5 mg, 81.0 mg, 81.5 mg, 82.0, or 82.5 mg. In one embodiment, the compound is present in the lyophilized formulation in an amount of between about 75.0 mg to about 82.5 mg, such as about 75.0 mg, about 75.5 mg, about 76.0 mg, about 76.5 mg, about 77.0 mg, about 77.5 mg, about 78.0 mg, about 78.5 mg, about 79.0 mg, about 79.5 mg, about 80.0 mg, about 80.5 mg, about 81.0 mg, about 81.5 mg, about 82.0, or about 82.5 mg.

In one embodiment, the compound is present in the lyophilized formulation in an amount of between about 100.0 mg to about 110.0 mg, such as 100.0 mg, 100.5 mg, 101.0 mg, 101.5 mg, 102.0 mg, 102.5 mg, 103.0 mg, 103.5 mg, 104.0 mg, 104.5 mg, 105.0 mg, 105.5 mg, 106.0 mg, 106.5 mg, 107.0 mg, 107.5 mg, 108.0 mg, 108.5 mg, 109.0 mg, 109.5 mg, or 110.0 mg. In one embodiment, the compound is present in the lyophilized formulation in an amount of between about 100.0 mg to about 110.0 mg, such as about 100.0 mg, about 100.5 mg, about 101.0 mg, about 101.5 mg, about 102.0 mg, about 102.5 mg, about 103.0 mg, about 103.5 mg, about 104.0 mg, about 104.5 mg, about 105.0 mg, about 105.5 mg, about 106.0 mg, about 106.5 mg, about 107.0 mg, about 107.5 mg, about 108.0 mg, about 108.5 mg, about 109.0 mg, about 109.5 mg, or about 110.0 mg.

In one embodiment, the compound is present in the lyophilized formulation in an amount of between about 200.0 mg to about 220.0 mg, such as 200.0 mg, 201.0 mg, 202.0 mg, 203.0 mg, 204.0 mg, 205.0 mg, 206.0 mg, 207.0 mg, 208.0 mg, 209.0 mg, 210.0 mg, 211.0 mg, 212.0 mg, 213.0 mg, 214.0 mg, 215.0 mg, 216.0 mg, 217.0 mg, 218.0 mg, 219.0 mg, or 220.0 mg. In one embodiment, the compound is present in the lyophilized formulation in an amount of between about 200.0 mg to about 220.0 mg, such as about 200.0 mg, about 201.0 mg, about 202.0 mg, about 203.0 mg, about 204.0 mg, about 205.0 mg, about 206.0 mg, about 207.0 mg, about 208.0 mg, about 209.0 mg, about 210.0 mg, about 211.0 mg, about 212.0 mg, about 213.0 mg, about 214.0 mg, about 215.0 mg, about 216.0 mg, about 217.0 mg, about 218.0 mg, about 219.0 mg, or about 220.0 mg.

In one embodiment, the compound is present in the lyophilized formulation in an amount of between about 300.0 mg to about 330.0 mg, such as 300.0 mg, 301.0, 302.0, 303.0 mg, 304.0 mg, 305.0 mg, 306.0 mg, 307.0 mg, 308.0 mg, 309.0 mg, 310.0 mg, 311.0 mg, 312.0 mg, 313.0 mg, 314.0 mg, 315.0 mg, 316.0 mg, 317.0 mg, 318.0 mg, 319.0 mg, 320.0 mg, 321.0 mg, 322.0 mg, 323.0 mg, 324.0 mg, 325.0 mg, 326.0 mg, 327.0 mg, 328.0 mg, 329.0 mg, or 330.0 mg. In one embodiment, the compound is present in the lyophilized formulation in an amount of between about 300.0 mg to about 330.0 mg, such as about 300.0 mg, about 301.0, about 302.0, about 303.0 mg, about 304.0 mg, about 305.0 mg, about 306.0 mg, about 307.0 mg, about 308.0 mg, about 309.0 mg, about 310.0 mg, about 311.0 mg, about 312.0 mg, about 313.0 mg, about 314.0 mg, about 315.0 mg, about 316.0 mg, about 317.0 mg, about 318.0 mg, about 319.0 mg, about 320.0 mg, about 321.0 mg, about 322.0 mg, about 323.0 mg, about 324.0 mg, about 325.0 mg, about 326.0 mg, about 327.0 mg, about 328.0 mg, about 329.0 mg, or about 330.0 mg.

### Kits

The present disclosure also contemplates kits comprising a lyophilized formulation comprising a compound of Formula (I) or (Ia) as described herein. The kits are generally in the form of a physical structure housing various components, as described below, and may be utilized, for example, in practicing the methods described above. The kit may include, for example, a syringe and a diluent for reconstitution as described above. When combination therapy is contemplated, the kit may contain the several agents separately or they may already be combined in the kit. Each component of the kit may be enclosed within an individual container, and all of the various containers may be within a single package. A kit of the present disclosure may be designed for conditions necessary to properly maintain the components housed therein (e.g., refrigeration or freezing).

A kit may contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

Labels or inserts can additionally include, or be incorporated into, a computer readable medium. In some embodiments, the actual instructions are not present in the kit, but means for obtaining the instructions from a remote source, e.g., via the internet, are provided.

### Experimental

The following examples are put forth so as to provide those of ordinary skill in the art with a complete disclosure and description of how to make and use the present disclosure, and are not intended to limit the scope of what the inventors regard as their disclosure, nor are they intended to represent that they are all of the experiments that may be performed. Efforts have been made to ensure accuracy with respect to numbers used (e.g., amounts, temperature, etc.), but some experimental errors and deviations should be accounted for.

### EXAMPLES

### Example 1: Preparation of Pre-Lyophilization Formulations

A compound of Formula (Ia), L-arginine, and phosphoric acid were added to sterile water and mixed until dissolved. Bulk solutions were transferred to a clean room for sterile filtration though a 0.22-micron pore size filter into a sterile vessel. Each sterile glass was filled to lyophilization depth. The vials were lyophilized according to the cycle in Table 1. The lyophilization chamber and vials were purged with nitrogen and the vials were stoppered. The vials were removed from the lyophilization chamber and a flip-off seal was placed on each vial and the seal was crimped. The vial was inspected for visual defects.

TABLE 1
Example Lyophilization Cycle

| | Temp | Time | Vacuum | Temp Ramp |
|---|---|---|---|---|
| Step # | (° C.) | (Minutes) | (mTorr) | (R)/Hold (H) |
| Thermal Treatment | | | | |
| 1 | −45 | 50 | NA | R |
| 2 | −45 | 240 | NA | H |
| Freeze Condenser | −45 | 10 | 100-300 | H |
| and Evacuate | | | | |
| Primary Drying | | | | |
| 1 | −25 | 30 | 100 | R |
| 2 | −25 | 1980 | 100 | H |
| 3 | +25 | 180 | 100 | R |
| Secondary | | | | |
| 1 | +25 | 300 | 100 | H |
| 2 | +25 | Until | NA | H |
| | | Stoppering | | |

### Example 2: Comparative Chemical and Physical Stability of Formulations

A compound of Formula (Ia) and various excipients were evaluated for chemical and physical suitability and stability. Based upon the results of these studies, three prototype formulations were selected for further evaluation of manufacturability and stability; the compositions of these prototypes are presented in Table 2. Formulations were evaluated for:
Pre-lyophilization solution clarity, content and purity for up to 5 days at 5° C.
Clarity, content, and purity of solutions reconstituted with either normal saline or sterile water for injection.
Stability of the reconstituted solutions for up to 5 days at 5° C. and 40° C.

Results of these studies are presented in the tables below. It was noted that the formulations containing amino acids demonstrated better physical stability as evidenced by the clarity scores of both pre-lyophilized solutions and reconstituted solutions.

TABLE 2
Composition of a Compound of Formula (Ia)
Lyophilized Powder Prototype Formulations

| | Batch 1 | Batch 2 | Batch 3 |
|---|---|---|---|
| Ingredient | mg/vial | mg/vial | mg/vial |
| Formula (Ia), neutral | 27.50 | 27.50 | 27.50 |
| L-Arginine USP | 37.13^{b} | | 8.25^{c} |
| L-Histidine USP | | | 22.04^{c} |
| Sodium hydroxide NF | | 4.17^{a} | |
| Phosphoric acid NF | 5.58^{b} | | |
| Sterile Water for Injection | qs. to 1.1 ml | qs. to 1.1 ml | qs. to 1.1 mL |

^{a}2.2 equivalents of sodium hydroxide
^{b}4.5 equivalents of L-arginine and 1.2 equivalents of phosphoric acid
^{c}1 equivalent of L-arginine and 3 equivalents of L-histidine

TABLE 3
Clarity Scores of Pre-Lyophilized and Reconstituted Solutions

| | | | 5° C./ | 5° C./ | 40° C./ | 40° C./ |
|---|---|---|---|---|---|---|
| Formulation | pH | Initial | 3 day | 5 day | 3 day | 5 day |
| Batch 1 PreLyo | 6.57 | 0 | 0 | 0-1 | — | — |
| Batch 2 PreLyo | 7.51 | 0 | 3 | 3 | — | — |
| Batch 3 PreLyo | 7.29 | 0 | 0 | 0-1 | — | — |
| Batch 1 Recon SWFI | — | 0 | 0 | 0-1 | 0 | 0-1 |
| Batch 1 Recon Saline | — | 0 | 0 | 0-1 | 0 | 0-1 |
| Batch 2 Recon SWFI | — | 0 | 1-2 | 3 | 3 | 3 |
| Batch 2 Recon Saline | — | 0 | 3 | 6 | 6 | 6 |
| Batch 3 SWFI | — | 0 | 0 | 0-1 | 0 | 0-1 |
| Batch 3 Saline | — | 0 | 0 | 0-1 | 0 | 0-1 |

PreLyo = pre-lyophilization; Recon = reconstitute; SWFI = sterile water for injection.
Formula (Ia) solutions at 25 mg/mL (41.3 mM).
Visual clarity scoring: 0 = clear; 6 = significantly hazy with particulate precipitation.

### HPLC Method

Column Agilent Zorbax Eclipse Plus C18; 4. ×100 mm, 3.5 μ; Lot #B17287
Detection Wavelength: 254 nm
Column Temp: 30±2° C.
Flow Rate: 1.0 mL/min
Sample Diluent: 20 mM Potassium Phosphate buffer pH 8.2, 25 μM EDTA
Sample Temperature: 20° C.
Mobile Phase A, MPA: 20 mM Potassium Phosphate buffer pH 8.2, 25 μM EDTA
Mobile Phase B, MPB: 100% Acetonitrile

### Mobile Phase Gradient

| Time (min) | % MPA | % MPB |
|---|---|---|
| 0.0 | 100 | 0 |
| 2.0 | 100 | 0 |
| 15.0 | 20 | 80 |
| 17.0 | 100 | 0 |
| 19.0 | 100 | 0 |

TABLE 4
Purity of Pre-lyophilized and Reconstituted Solutions: Initial and
After Storage for 3 and 5 days at 5° c. and 40° c.

| | Initial Purity | 3 days, | 5 days, | 3 days, | 5 days, |
|---|---|---|---|---|---|
| Sample | (% area) | 5° C. | 5° C. | 40° C. | 40° C. |
| Batch 1 | | | | | |
| Recon SWFI | 97.53 | 97.51 | 97.64 | 97.73 | 97.57 |
| Recon Saline | 97.61 | 97.58 | 97.69 | 97.72 | 97.71 |
| Batch 2 | | | | | |
| Recon SWFI | 97.67 | 97.70 | 97.62 | 97.73 | 97.71 |
| Recon Saline | 97.66 | 97.71 | 97.72 | 97.80 | 97.76 |
| Batch 3 | | | | | |
| Recon SWFI | 97.66 | 97.65 | 97.64 | 97.79 | 97.72 |
| Recon Saline | 97.64 | 97.73 | 97.74 | 97.77 | 97.69 |

Recon = reconstituted solution; SWFI = sterile water for injection.
Samples reconstituted to a theoretical concentration of 25 mg/mL.

### Example 3: Lyophilization of Formulations Containing Bulking Agents

The cake appearance of lyophiles, reconstitution parameters, and stability of reconstituted solutions were evaluated for formulations containing a compound of Formula (Ia), arginine, phosphoric acid, and various co-solutes (i.e., bulking agents). The vial configurations of the pre-lyophilized formulations are summarized in Table 5 below.

TABLE 5
Configuration of Vials Containing Formulations With Co-Solutes

| Formula (Ia) pre-lyophilized | |
|---|---|
| solution ingredients | Configuration (mg/vial) |
| Formula (Ia), neutral | 107.50 |
| Arginine, USP | 145.34 |
| Phosphoric Acid, NF | 21.84 |
| Co-solute | Various ratios (see Table 7) |
| 0.1N NaOH/Phosphoric Acid | q.s. to pH 7.2 to 7.4 |
| Sterile Water for Injection | q.s. to fill volume/vial |
| Vial | 10 mL vial (4.3 mL fill) |
| | 20 mL vial (4.3 mL fill) |
| | 20 mL vial (6.0 mL fill) |
| Formula (Ia) concentration in | 25 mg/mL (4.3 mL fill) |
| pre-lyophilization solution | 17.9 mg/mL (6 mL fill) |

The formulations were lyophilized according to the lyophilization cycles summarized for the various co-solutes in Table 6 below. The primary drying and secondary drying steps of the lyophilization cycle were both carried out under reduced pressure. Typical drying times for the primary drying phase and the secondary drying phase were about 36-40 hours and 7-10 hours, respectively.

TABLE 6
Lyophilization Cycles for Various Co-Solutes

| | | Primary | Secondary | Total |
|---|---|---|---|---|
| | Annealing | Drying | Drying | Cycle |
| | Temp | Temp | Temp | Time |
| Co-Solute | (° C.) | (° C.)* | (° C.)* | (hrs) |
| No Added | −10 | −15 | 25 | 72 |
| Co-Solute | | | | |
| Kleptose | Not needed | −10 | 25 | 50 |
| (HPBCD) | | | | |
| Glycine | −15 | −15 | 25 | 66 |
| (Lots A-D) | | | | |
| Glycine | −10 | −15 | 25 | 72 |
| (Lot E) | | | | |
| Dextran 40 | Not needed | −10 | 25 | 52 |
| Mannitol | −10 | −15 | 25 | 73 |
| (Lots A-D) | | | | |
| Mannitol | −10 | −15 | 25 | 72 |
| (Lot E) | | | | |

The reconstitution time and stability of the reconstituted solutions was assessed. Reconstitution time, solution pH, and osmolality for the various formulations are summarized Table 7 below.

TABLE 7
Reconstitution Results For Formulations Containing Co-Solutes

| | | | | | | | |
|---|---|---|---|---|---|---|---|
| | Co- | | | | | | |
| | solute | | | | | | |
| | level | | Vial | | Recon | | |
| Batch | (mass | Fill | Size | | Time | Solution | Osmolality |
| No. | %) | (mL) | (mL) | SWFI Added (mL) | (sec) | pH | (mOsm/kg) |

Kleptose Co-Solute

| | | | | | | | |
|---|---|---|---|---|---|---|---|
| A1 | 50 | 4.3 | 10 | 4.3 | 28 | 7.33 | 211 |
| A2 | 50 | 4.3 | 20 | 4.3 | 48 | 7.33 | 212 |
| B1 | 33 | 4.3 | 10 | 4.3 | 30 | 7.33 | 190 |
| B2 | 33 | 4.3 | 20 | 4.3 | 36 | 7.32 | 188 |
| C1 | 20 | 4.3 | 10 | 4.3 | 26 | 7.33 | 174 |
| C2 | 20 | 4.3 | 20 | 4.3 | 26 | 7.33 | 174 |
| D | 50 | 6 | 20 | 4.3 | 28 | 7.32 | 192 |
| E | 33 | 6 | 20 | 4.3 | 17 | 7.32 | 192 |

Glycine Co-Solute

| | | | | | | | |
|---|---|---|---|---|---|---|---|
| A | 50 | 4.3 | 20 | 4.3 | 22 | 7.18 | 923 |
| B1 | 42 | 4.3 | 10 | 4.3 | 17 | 7.17 | 710 |
| B2 | 42 | 4.3 | 20 | 4.3 | 24 | 7.17 | 716 |
| C | 50 | 6 | 20 | 4.3 | 21 | 7.13 | 932 |
| D | 42 | 6 | 20 | 4.3 | 17 | 7.17 | 710 |
| E | 42 | 4.3 | 20 | 4.3 | 22 | 7.24 | 713 |

Dextran 40 Co-Solute

| | | | | | | | |
|---|---|---|---|---|---|---|---|
| A1 | 50 | 4.3 | 10 | 4.3 | 178 | 7.27 | 178 |
| A2 | 50 | 4.3 | 20 | 4.3 | 171 | 7.27 | 171 |
| B1 | 33 | 4.3 | 10 | 4.3 | 68 | 7.32 | 166 |
| B2 | 33 | 4.3 | 20 | 4.3 | 62 | 7.32 | 164 |
| C1 | 20 | 4.3 | 10 | 4.3 | 49 | 7.33 | 164 |
| C2 | 20 | 4.3 | 20 | 4.3 | 42 | 7.33 | 165 |
| D | 50 | 6 | 20 | 4.3 | 82 | 7.32 | 170 |
| E | 33 | 6 | 20 | 4.3 | 72 | 7.33 | 165 |

Mannitol Co-Solute

| | | | | | | | |
|---|---|---|---|---|---|---|---|
| A1 | 60 | 4.3 | 10 | 4.3 | 109 | 7.25 | 672 |
| A2 | 60 | 4.3 | 20 | 4.3 | 81 | 7.26 | 692 |
| B1 | 63.6 | 4.3 | 10 | 4.3 | 159 | 7.26 | 794 |
| B2 | 63.6 | 4.3 | 20 | 4.3 | 67 | 7.25 | 797 |
| C | 60 | 6 | 20 | 4.3 | 64 | 7.72 | 685 |
| D | 63.6 | 6 | 20 | 4.3 | 74 | 7.26 | 790 |
| E | 55.6 | 4.3 | 20 | 4.3 | 20 | 7.25 | 616 |

No Added Co-Solute

| | | | | | | | |
|---|---|---|---|---|---|---|---|
| A | 0 | 4.3 | 22 | 4.3 | 15 | 7.26 | 161 |

Fill volumes correspond to the volume of solvent added prior to lyophilization; SWFI Added corresponds to the volume of SWFI added for reconstitution; Recon = reconstitution; SWFI = sterile water for injection.

As compared to the lyophile lacking a bulking agent, all lyophiles containing a bulking agent exhibited less cracking, brittleness, and shrinkage.

It was also observed that the type of vial used to lyophilize the formulations also played a role in the appearance of the resulting cake. The lyophiles made using SCHOTT TopLyo® vials formed intact cakes with no fragmentation when mannitol, kleptose and dextran 40 were used as bulking agents.

All tested lyophiles reconstituted rapidly to form clear solutions.

The stability of selected reconstituted solutions was assessed by high-performance liquid chromatography (HPLC) for solutions stored at 40° C. at 75% relative humidity (RH) for 2 and 4 weeks, and solutions stored at −20° C. for two weeks. The results are summarized in Table 8 below.

### HPLC Method

Column Waters Xbridge Shield RP18; 4.6×150 mm, 3.5 μm, Detection Wavelength: 305 nm; Column Temp: 30±2° C.; Injection Volume: 5 μL; Flow Rate: 1.0 mL/min; Run Time: 50 minutes; Sample Diluent: 20 mM Potassium Phosphate buffer pH 10.0±0.05; Retention Time: ˜13 minutes; Sample Temperature: 5.0±1° C.; Needle Rinse: Acetonitrile: Water (50:50); Mobile Phase A (MPA): 10% acetonitrile: 90% 20 mM potassium phosphate buffer pH 10.0±0.05; Mobile Phase B (MPB): 65% Acetonitrile: 35% 20 mM potassium phosphate, pH 10.0±0.05

### Mobile Phase Gradient

| Time (min) | % MPA | % MPB |
|---|---|---|
| 0.0 | 100 | 0 |
| 2.0 | 100 | 0 |
| 10.0 | 70 | 30 |
| 14.0 | 70 | 30 |
| 30.0 | 0 | 100 |
| 39.0 | 0 | 100 |
| 40.0 | 100 | 0 |
| 50.0 | 100 | 0 |

TABLE 8
Purity of Reconstituted Solutions after storage at −20° C. for
2 weeks, 40° C. for 2 weeks, and 40° C. for 4 weeks

| | −20° C. | 40° C. @ 75% | 40° C. @ 75% |
|---|---|---|---|
| Condition | (2 weeks) | RH (2 weeks) | RH (4 weeks |
| Bulking Agent | Formula (Ia) Area % | | |
| Kleptose (32 mg/mL) | 97.89 | 97.86 | 97.81 |
| Glycine (46.5 mg/mL) | 97.90 | 97.94 | 97.93 |
| Dextran 40 | 97.85 | 97.87 | 97.86 |
| (32.0 mg/mL) | | | |
| Mannitol (96.0 mg/mL) | 97.87 | 97.87 | 97.87 |

All formulations demonstrated chemical stability, and had similar impurity profiles as determined by HPLC when stored at −20° C. for 2 weeks and 40° C. at 75% RH for 4 weeks. All tested solutions were also physically stable, and remained clear for the duration of the study.

### Example 4: Exemplary Protocol for the Preparation of a Bulk Quantity of Lyophilized Formulation According to the Disclosure

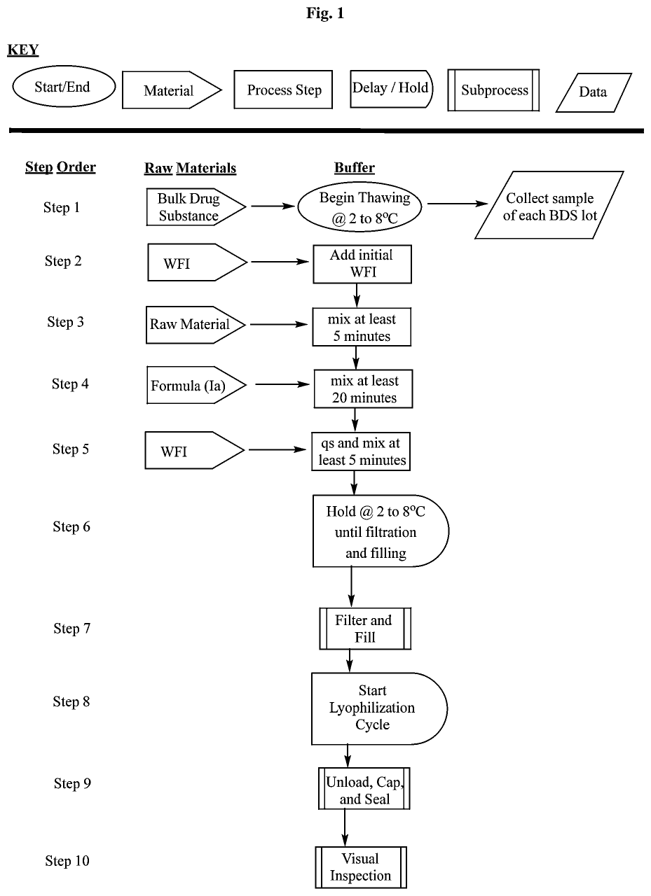

The components of the lyophilized formulation are summarized in Table 9 below. The process for bulk lyophilization is described in FIG. 1. The parameters of the freeze-drying process are summarized in Table 10 below.

TABLE 9
Exemplary Components of Lyophilized Formulation

| Ingredient | mg/vial |
|---|---|
| Formula (Ia), neutral | 107.5 |
| Arginine USP | 145.34 |
| Phosphoric acid NF | 21.84 |
| Mannitol | 344 |
| 0.1N NaOH/Phosphoric acid | qs to pH 7.2 to 7.4 |
| SWFI | qs 4.3 mL |

TABLE 10
Freeze-Drying Process Parameters

| Process Step | Description |
|---|---|
| Loading | 152 vials containing the pre-lyophilized solutions were |
| | loaded on to the freeze-dryer shelf at room temperature. |
| Freezing | Shelf was cooled to 5° C. and held for 30 min |
| | Shelf was next cooled to −45° C. at 0.5° C./min and |
| | held at −45° C. for 3 hours. |
| Annealing | The shelf was heated to −10° C., over 50 minutes, and |
| | maintained at −10° C. for 4 hours. |
| | The shelf was cooled back to −45° C., over 70 minutes |
| | and maintained at −45° C. for 2 hours. |
| Primary | The shelf temperature was increased to −15° C., over |
| Drying | 70 minutes and the chamber evacuated to 150 mTorr |
| | to start primary drying and maintained for 2150 min. |
| | Probe temperatures increased to match shelf |
| | temperature after ~1750-2000 minutes |
| Temperature | Shelf temperature was ramped to 30° C., over 200 |
| Ramp | minutes. |
| Secondary | Secondary drying was carried out with a shelf |
| Drying | temperature of +30° C., at ~150 mTorr chamber |
| | pressure for 4 hours. |
| Stoppering | The chamber was backfilled with dry nitrogen and the |
| | vials stoppered under nitrogen atmosphere at ~700 |
| | Torr |

### Example 5: A Phase II Study to Evaluate Zimberelimab-Based Combinations in Participants With Advanced Non-small Cell Lung Cancer

A phase II open label platform study will be used to investigate the safety and efficacy of zimberelimab with other investigational products in participants with non-small cell lung cancer. Sub-study A will enroll treatment-naive, PD-L1 high, metastatic NSCLC patients without actionable genomic aberrations. Sub-study B will enroll treatment-naive, metastatic NSCLC patients without actionable genomic aberrations, but without restriction to PD-L1 status. PD-L1 high status may be assessed by tumor proportion score (TPS))>50% by PharmDx 22C3 (Dako) or tumor cell (TC) >50% by SP263 (Ventana). Sub-Study C will enroll metastatic NSCLC patients who have documented disease progression on anti-PD-L1 and platinum-based chemotherapy in one or two lines of prior therapy and do not have known actionable genomic aberrations. Arm A1: zimberelimab 360 mg Q3W+domvanalimab 5 mg/kg Q3W; Arm A2: zimberelimab 360 mg Q3W+domvanalimab 15 mg/kg Q3W; Arm A3: zimberelimab 480 mg Q4W+domvanalimab 1600 mg Q4W+quemliclustat 100 mg Q2W; Arm B1: zimberelimab 360 mg Q3W+quemliclustat 50 mg QW+platinum doublet chemotherapy; Arm B2: zimberelimab 360 mg Q3W+domvanalimab 1200 mg Q3W+platinum doublet chemotherapy; Arm B3: zimberelimab 360 mg Q3W+domvanalimab 1200 mg Q3W+quemliclustat 50 mg QW+platinum doublet chemotherapy; Arm C1: zimberelimab 360 mg Q3W+domvanalimab 1200 mg Q3W+docetaxel; Arm C2: zimberelimab 360 mg Q3W+quemliclustat 300 mg Q3W+docetaxel. Primary endpoints to be assessed include objective response rate, safety and tolerability. Secondary endpoints include progression-free survival, duration of response, overall survival, and PK.

### Example 5-1: Q3W Dosing of the Compound of Formula (Ia)

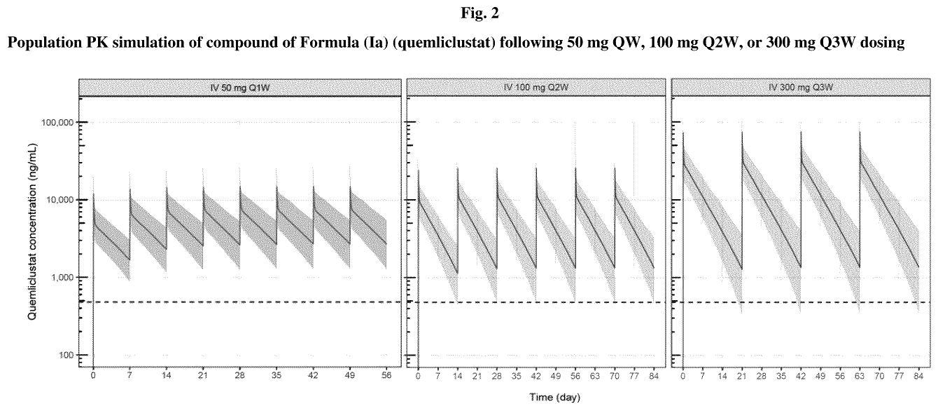

In the study set forth above a formulation according to this disclosure may be given at 300 mg 3QW. Preliminary population PK/PD modeling suggests that in order to maintain steady state Ctrough (Ctrough,ss) comparable to 50 mg QW, or 100 mg Q2W, substantially higher doses are required when extending the dosing interval. A 300 mg Q3W dose level may yield median steady state Ctrough equivalent to that for 50 mg QW, and 100 mg Q2W for achieving >90% inhibition of CD73 enzyme activity in majority of participants. FIG. 2 shows predicted population PK/PD following 50 mg QW, 100 mg Q2W, or 300 mg Q3W dosing. The simulations were performed based on a population pharmacokinetic/pharmacodynamic model developed using pharmacokinetic and pharmacodynamic data collected from multiple clinical trials.

Particular embodiments of this disclosure are described herein. Upon reading the foregoing, description, variations of the disclosed embodiments may become apparent to individuals working in the art, and it is expected that those skilled artisans may employ such variations as appropriate. Accordingly, it is intended that the disclosure be practiced otherwise than as specifically described herein, and that the disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All publications, patent applications, accession numbers, and other references cited in this specification are herein incorporated by reference as if each individual publication or patent application were specifically and individually indicated to be incorporated by reference.

1. A lyophilized formulation comprising a compound of Formula (I) 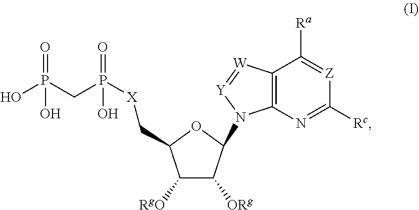
or a pharmaceutically acceptable salt, hydrate, or solvate thereof, wherein,
W is selected from the group consisting of CR^{e} and N;
X is selected from the group consisting of O, CH₂, and S;
each of Y and Z is independently selected from the group consisting of CH and N:
R^{g} is H or the two R^{g} groups are combined to form an acetonide;
R^{a} is selected from the group consisting of NH₂, NHR¹, and NR¹R²;
R^{c} is selected from the group consisting of H, halogen, haloalkyl, NH₂, NHR³, NR³R⁴, R³, OH, OR³, SR³, SO₂R³, —X¹—NH₂, —X¹—NHR³, —X¹—NR³R⁴, —X¹—OH, —X¹—OR³, —X¹—SR³, and —X¹—SO₂R³;
R^{e} is selected from the group consisting of H, halogen, and optionally substituted C₁-C₆ alkyl;
each X¹ is C₁-C₄ alkylene; and
each R¹, R², R³, and R⁴ is independently selected from the group consisting of optionally substituted C₁-C₁₀ alkyl, optionally substituted C₃-C₇ cycloalkyl, optionally substituted C₃-C₇ cycloalkyl C₁-C₄ alkyl, optionally substituted 4-7 membered heterocycloalkyl, optionally substituted 4-7 membered heterocycloalkyl C₁-C₄ alkyl, optionally substituted aryl, optionally substituted aryl C₁-C₄ alkyl, optionally substituted heteroaryl, and optionally substituted heteroaryl C₁-C₄ alkyl, or when R¹ and R² or R³ and R⁴ are attached to the same nitrogen, they combine to form a 4- to 7-membered heterocyclic ring;
and one or more amino acids.

2. The lyophilized formulation of claim 1, wherein X is O.

3. The lyophilized formulation of claim 1, wherein R^{g} is H.

4. The lyophilized formulation of claim 1, wherein R^{a} is NHR¹ and R¹ is selected from the group consisting of optionally substituted aryl C₁-C₄alkyl and optionally substituted heteroaryl C₁-C₄alkyl.

5. The lyophilized formulation of claim 4, wherein R^{a} is NHR¹ and R¹ is optionally substituted aryl C₁-C₄alkyl.

6. The lyophilized formulation of claim 1, wherein R^{c} is selected from the group consisting of H, halogen, and haloalkyl.

7. The lyophilized formulation of claim 6, wherein R^{c} is halogen.

8. The lyophilized formulation of claim 1, wherein R^{e} H.

9. The lyophilized formulation of claim 1, wherein the compound of Formula (I) has a structure selected from the group consisting of 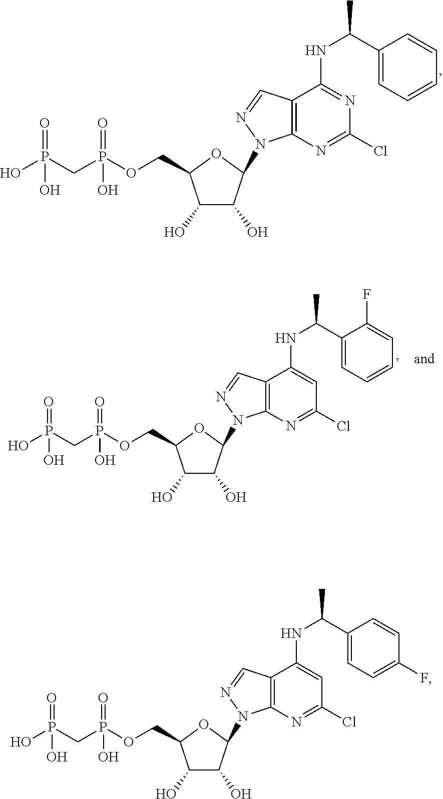 [SMILES: C[C@H](Nc1nc(Cl)nc2c1cnn2[C@@H]1O[C@H](COP(=O)(O)CP(=O)(O)O)[C@@H](O)[C@H]1O)c1ccccc1 ; C[C@H](Nc1cc(Cl)nc2c1cnn2[C@@H]1O[C@H](COP(=O)(O)CP(=O)(O)O)[C@@H](O)[C@H]1O)c1ccccc1F ; C[C@H](Nc1cc(Cl)nc2c1cnn2[C@@H]1O[C@H](COP(=O)(O)CP(=O)(O)O)[C@@H](O)[C@H]1O)c1ccc(F)cc1]
or a pharmaceutically acceptable salt thereof.

10. The lyophilized formulation of claim 1, wherein the compound of Formula (I) has a structure according to Formula (Ia): 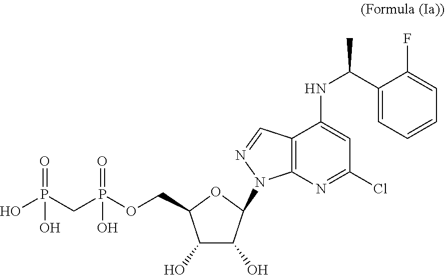 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]
or a pharmaceutically acceptable salt thereof.

11. The lyophilized formulation of claim 1, wherein, the one or more amino acids comprises arginine, lysine, histidine, tryptophan, cysteine, or a combination thereof

12. The lyophilized formulation of claim 1, further comprising a pH-adjusting agent.

13. The lyophilized formulation of claim 12, wherein the pH-adjusting agent is phosphoric acid.

14. The lyophilized formulation of claim 1, further comprising a bulking agent.

15. The lyophilized formulation of claim 14, wherein the bulking agent is mannitol, glycine, hydroxypropyl-Beta-cyclodextrin (HPBCD), or dextran.

16. The lyophilized formulation of claim 1, wherein the molar ratio of the compound of Formula (I) to the one or more amino acids is from about 1:2 to about 1:7.

17. The lyophilized formulation of claim 1, wherein the molar ratio of the compound of Formula (I) to the one or more amino acids is from about 1:4 to about 1:5.

18. The lyophilized formulation of claim 1 wherein the compound is present in an amount of between about 25 mg to about 27.5 mg.

19. The lyophilized formulation of claim 1, wherein the compound is present in an amount of between about 50 mg to about 55 mg.

20. The lyophilized formulation of claim 1, wherein the compound is present in an amount of between about 75 mg to about 82.5 mg.

21. The lyophilized formulation of claim 1, wherein the compound is present in an amount of about 100 mg to about 110 mg.

22. The lyophilized formulation of claim 1, wherein the compound is present in an amount of about 200 mg to about 325 mg.

23. The lyophilized formulation of claim 1, comprising a compound of Formula (Ia) 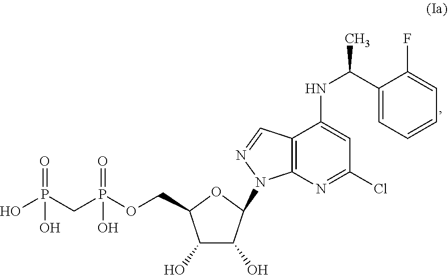 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]
or a pharmaceutically acceptable salt thereof, and one or more amino acids selected from arginine, lysine, histidine, tryptophan, cysteine, and combinations thereof in an amount greater than the stoichiometric amount of the compound of Formula (Ia).

24. The lyophilized formulation of claim 1, comprising
about 15% to about 20% by weight of a compound of Formula (Ia) 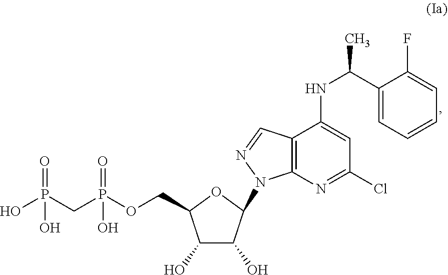 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]
or a pharmaceutically acceptable salt, hydrate, or solvate thereof,
about 20% to about 35% by weight of one or more amino acids;
about 2% to about 5% by weight of phosphoric acid; and
about 40% to about 60% by weight of a bulking agent;
wherein percentage by weight (% by weight) is based on the total weight of the lyophilized formulation.

25. The lyophilized formulation of claim 24, wherein the bulking agent is kleptose, dextran, mannitol, or glycine.

26. The lyophilized formulation of claim 24, comprising
about 17.4% by weight of the compound of Formula (Ia), or a pharmaceutically acceptable salt, hydrate, or solvate thereof,
about 23.4% by weight of arginine;
about 3.5% by weight of phosphoric acid; and
about 55.6% by weight of mannitol;
wherein percentage by weight is based on the total weight of the lyophilized formulation.

27. The lyophilized formulation of claim 24, comprising
about 22.6% by weight of the compound of Formula (Ia), or a pharmaceutically acceptable salt, hydrate, or solvate thereof,
about 30.6% by weight of arginine;
about 4.6% by weight of phosphoric acid; and
about 42.1% by weight of glycine;
wherein percentage by weight is based on the total weight of the lyophilized formulation.

28. The lyophilized formulation of claim 1, comprising
about 35% to about 45% by weight of a compound of Formula (Ia) 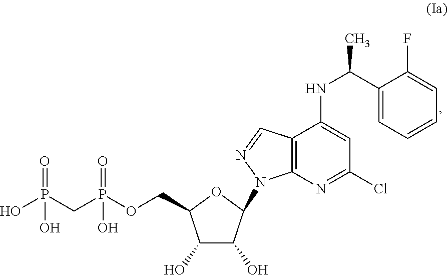 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]
or a pharmaceutically acceptable salt, hydrate, or solvate thereof,
about 45% to about 55% by weight of one or more amino acids;
about 5% to about 10% by weight of phosphoric acid; and
wherein percentage by weight (% by weight) is based on the total weight of the lyophilized formulation.

29. The lyophilized formulation of claim 28, comprising
about 39.2% by weight of the compound of Formula (Ia), or a pharmaceutically acceptable salt, hydrate, or solvate thereof,
about 52.9% by weight of arginine; and
about 7.9% by weight of phosphoric acid;
wherein percentage by weight is based on the total weight of the lyophilized formulation.

30. The lyophilized formulation of claim 1, wherein the lyophilized formulation is in a vial and comprises
about 107.5 mg of a compound of Formula (Ia) 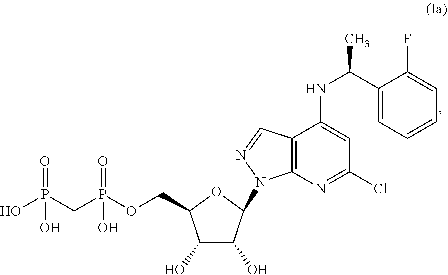 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]
or a pharmaceutically acceptable salt, hydrate, or solvate thereof,
about 145.34 mg of arginine;
about 21.8 mg of phosphoric acid; and
about 344 mg of mannitol.

31. The lyophilized formulation of claim 1, wherein the lyophilized formulation is in a vial and comprises
about 107.5 mg of a compound of Formula (Ia) 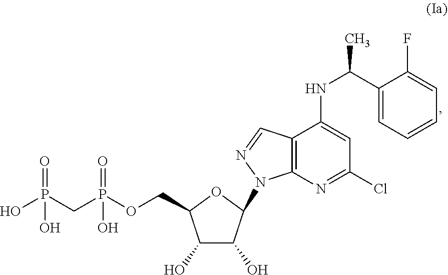 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]
or a pharmaceutically acceptable salt, hydrate, or solvate thereof,
about 145.34 mg of arginine;
about 21.8 mg of phosphoric acid; and
about 200 mg of glycine.

32. The lyophilized formulation of claim 1, wherein the lyophilized formulation is in a vial and comprises
about 27.5 mg of a compound of Formula (Ia) 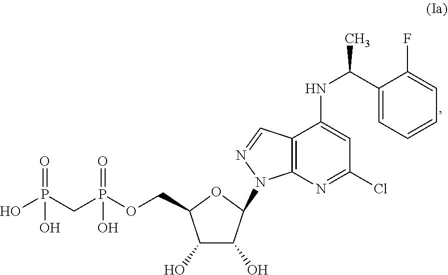 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]
or a pharmaceutically acceptable salt, hydrate, or solvate thereof,
about 37.1 mg of arginine; and
about 5.6 mg of phosphoric acid.

33. The lyophilized formulation of claim 1, wherein the lyophilized formulation is in a vial and comprises
about 107.5 mg of a compound of Formula (Ia) 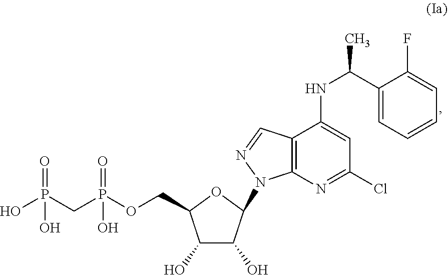 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]
or a pharmaceutically acceptable salt, hydrate, or solvate thereof,
about 145.1 mg of arginine; and
about 21.8 mg of phosphoric acid.

34. An aqueous formulation comprising a compound of Formula (I) 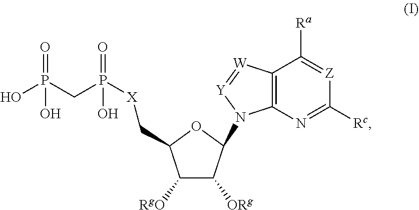
or a pharmaceutically acceptable salt, hydrate, or solvate thereof, wherein,
W is selected from the group consisting of CR^{e} and N;
X is selected from the group consisting of O, CH₂, and S;
each of Y and Z is independently selected from the group consisting of CH and N:
R^{g} is H or the two R^{g} groups are combined to form an acetonide;
R^{a} is selected from the group consisting of NH₂, NHR¹, and NR¹R²;
R^{c} is selected from the group consisting of H, halogen, haloalkyl, NH₂, NHR³, NR³R⁴, R³, OH, OR³,SR³, SO₂R³, —X¹—NH₂, —X¹—NHR³, —X¹—NR³R⁴, —X¹—OH, —X¹—OR³, —X¹—SR³, and —X¹—SO₂R³;
R^{e} is selected from the group consisting of H, halogen, and optionally substituted C₁-C₆ alkyl;
each X¹ is C₁-C₄ alkylene; and
each R¹, R², R³, and R⁴ is independently selected from the group consisting of optionally substituted C₁-C₁₀ alkyl, optionally substituted C₃-C₇ cycloalkyl, optionally substituted C₃-C₇ cycloalkyl C₁-C₄ alkyl, optionally substituted 4-7 membered heterocycloalkyl, optionally substituted 4-7 membered heterocycloalkyl C₁-C₄ alkyl, optionally substituted aryl, optionally substituted aryl C₁-C₄ alkyl, optionally substituted heteroaryl, and optionally substituted heteroaryl C₁-C₄ alkyl, or when R¹ and R² or R³ and R⁴ are attached to the same nitrogen, they combine to form a 4- to 7-membered heterocyclic ring;
one or more amino acids and optionally a pH-adjusting agent in an amount sufficient to adjust the pH of the solution to between about 6 and about 8.

35. An aqueous formulation comprising
about 15% to about 20% by weight of a compound of Formula (Ia) 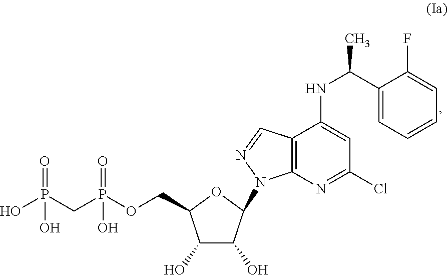 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]
or a pharmaceutically acceptable salt, hydrate, or solvate thereof,
about 20% to about 35% by weight of one or more amino acids;
about 2% to about 5% by weight of phosphoric acid; and
about 40% to about 60% by weight of a bulking agent;
wherein percentage by weight (% by weight) is based on the total weight of a lyophilized formulation that is dissolved in water to form the aqueous formulation.

36.-38. (canceled)

39. An aqueous formulation comprising
about 107.5 mg of a compound of Formula (Ia) 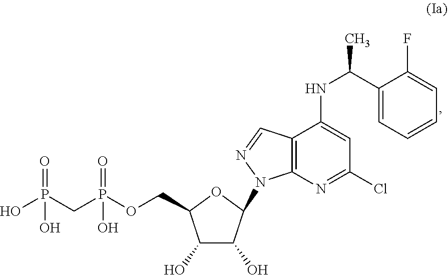 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]
or a pharmaceutically acceptable salt, hydrate, or solvate thereof,
about 145.34 mg of arginine;
about 21.8 mg of phosphoric acid; and
about 344 mg of mannitol.

40. An aqueous formulation comprising
about 107.5 mg of a compound of Formula (Ia) 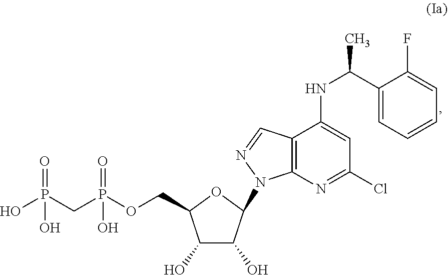 [SMILES: C[C@H](Nc1cc(Cl)nc2c1cnn2[C@@H]1O[C@H](COP(=O)(O)CP(=O)(O)O)[C@@H](O)[C@H]1O)c1ccccc1F]
or a pharmaceutically acceptable salt, hydrate, or solvate thereof,
about 145.34 mg of arginine;
about 21.8 mg of phosphoric acid; and
about 200 mg of glycine.

41.-45. (canceled)

46. A method of treating a disease, disorder, or condition mediated at least in part by CD73, said method comprising:
a) reconstituting the lyophilized formulation according to claim 1 with a diluent to form a reconstituted solution; and
b) administering a therapeutically effective amount of the reconstituted solution to a subject in need thereof.

47.-74. (canceled)

75. A process for preparing a lyophilized formulation of claim 1, comprising
(i) preparing a bulk solution by dissolving the compound of Formula (Ia), arginine, phosphoric acid, and a bulking agent in sterile water;
(ii) optionally adjusting the pH of the bulk solution by adding a pH-adjusting agent;
(iii) filtering the bulk solution into sterilized vials in a clean room;
(iv) loading the vials into a freeze-dryer;
(v) subjecting the vials to freeze-thaw cycles; and
(vi) subjecting the vials to primary and secondary drying under reduced pressure, thereby obtaining the lyophilized formulation.

76.-102. (canceled)
